(19) United States
(12) Patent Application Publication
(10) Pub. No.: US 2022/0090010 A1
(43) Pub. Date: Mar. 24, 2022

(54) METHOD OF PRODUCING ENTERIC NEURONS AND USES THEREOF

(71) Applicant: THE REGENTS OF THE UNIVERSITY OF CALIFORNIA, Oakland, CA (US)

(72) Inventor: Faranak Fattahi, San Francisco, CA (US)

(21) Appl. No.: 17/416,962

(22) PCT Filed: Dec. 23, 2019
(86) PCT No.: PCT/US2019/068447

§ 371 Date: Jun. 21, 2021

Related U.S. Application Data
(60) Provisional application No. 62/783,795, filed on Dec. 21, 2018.

(51) Int. Cl. C12N 5/0793

(52) U.S. Cl. CPC C12N 5/0619; C12N 2500/25; C12N 2500/44; C12N 2500/46; C12N 2500/90; C12N 2501/115; C12N 2533/90; C12N 2501/392; C12N 2501/727; C12N 2506/02; C12N 2506/45; C12N 2513/00; C12N 2533/52; C12N 2501/385

(57) ABSTRACT

The present disclosure relates generally to methods and systems of producing enteric neurons from pluripotent stem cells under fully defined conditions. The enteric neural crest cells and enteric neurons produced by the disclosed methods find applications as models of the enteric nervous system, tools for high-throughput screening of potential therapeutics for treatment of enteric neuropathies, and in regenerative medicine.

### CROSS-REFERENCE TO RELATED APPLICATIONS

The application is a National Stage application filed under 35 U.S.C. § 371 of International Application No. PCT/US2019/068447, filed on Dec. 23, 2019, which claims priority to U.S. Provisional Application No. 62/783,795, filed on Dec. 21, 2018, which are incorporated by reference in their entireties.

### SEQUENCE LISTING

This application is submitted with a Sequence Listing text file in ASCII format, which serves as both the computer readable form (CFR) and the paper copy as required under 37 C.F.R. § 1.821. Said Sequence Listing text file is: entitled “37944_0013P1_ST25.txt”, 101,756 bytes in size, created on Jun. 19, 2021, and incorporated by reference in its entirety.

### TECHNOLOGY FIELD

The present disclosure relates generally to methods of culturing pluripotent stem cells in defined conditions, inducing the pluripotent stem cells to differentiate into enteric neural crest cells, then the neural crest cells are cultured to produced spheroids, which in turn are induced to differentiate into enteric neurons. The resulting enteric neurons are suitable for screening potential therapeutic agents for the treatment of enteric neuropathies such as gastroparesis, esophageal achalasia, chronic intestinal pseudo-obstruction, and hypertrophic pyloric stenosis, and applications in regenerative medicine.

### BACKGROUND

During embryogenesis, neural crest (NC) induction occurs at the interface of the non-neuronal ectoderm and the folding neural plate as a result of bone morphogenic protein (BMP), fibroblast growth factor (FGF), and Wnt signaling pathway activity (1). During neurulation, dorsally localized NC cells delaminate and migrate away from the newly formed neural tube. Migratory NC cells proliferate and act as progenitors for a remarkable diversity of cell types including various populations of peripheral neurons and glia, melanocytes, endocrine cells and mesenchymal precursor cells (1-3). In the developing embryo, the neural crest shows an anterior-posterior spatial organization associated with the expression of regionally specific HOX genes. Distinct functional regions include the cranial NC, vagal NC, trunk NC and sacral NC located anteriorly to posteriorly respectively (FIG. 1).

While the enteric nervous system (ENS) is generated from both the vagal and sacral NC, vagal NC lineages positive for HOXB3 (4) and HOXB5 (5) migrate most extensively to colonize the entire length of the bowel (6) (arrows in FIG. 1). Upon inclusion into the foregut, vagal NC cells display enteric neural crest (ENC) identity characterized by the expression of SOX10, PHOX2B, EDNRB, and ASCL1. Colonization of the intestinal tract by the ENC has been depicted as a rostrocaudally moving wave of proliferative multipotent ENS progenitors (7). By week seven of embryogenesis in humans, migratory ENC cells will reach the terminal hindgut (8). Failure of ENC migration to the caudal regions of the bowel can result in congenital aganglionosis of the colon, a disorder known as Hirschsprung's disease.

Post migratory ENC cells will commit to neuronal fates, a differentiation step associated with the downregulation of SOX10, sustained expression of EDNRB, ASCL1 and PHOX2B, and upregulation of pan neuronal markers such as TUJ1 (9). ENC progenitors further differentiate to establish ganglia located between the circular and longitudinal layers of enteric smooth muscle, forming the myenteric plexus. Recent spatiotemporal analysis of the murine ENS has shown that ENC progenitors within the myenteric plexus proliferate along the serosa-mucosal axis to subsequently form the ganglia of the submucosal plexus (10). Together, the myenteric and submucosal plexi will establish the neuronal circuitry of the functional ENS.

Due to the capacity of the NC to undergo an extensive range of cell fate decisions, protocols seeking to optimize NC induction and subtype specification from hPSCs have been an important focus of research (11-13). Such hPSC-based NC protocols commonly rely on a variation of the dual SMAD signaling inhibition protocol for neural induction, combined with the temporal activation of WNT signaling (12-14). However, such methods often involve the use of poorly defined culture components such as serum, BSA fractions, and other animal-derived products, that may affect the reliability and reproducibility of NC induction (e.g. Comparative Example 2). Accordingly, the inventors and others have reported protocols that use fully defined, xeno-free culture conditions for the reliable induction of cranial NC from hPSCs (15, 16).

The spatial and temporal transience of the ENC has been a major factor in limiting access to primary cells, particularly from human embryonic or fetal tissue samples. As a result, studying the developing ENS has largely relied upon studies in murine models. Work with such murine models resulted in the discovery of growth factors involved in the proliferation and differentiation of EN precursors, such as Neurotrophin-3 (NT-3) and glial cell line-derived neurotrophic factor (GDNF) (17, 18) among others. More recent single cell transcriptomics analysis of the developing murine ENS have revealed novel molecular states of lineally and functionally related ENS progenitors (10). An appreciable conservation of the transcriptional processes underpinning ENS development across mammals (19) supports the application of these factors to direct hPSC-derived ENC cells towards neurogenic commitments and may help further guide the identification, characterization and derivation of human enteric neuronal subtype lineages.

Therefore, there remains a need for novel protocols for derivation of enteric neurons (ENs) from hPSCs and a basis for modeling ENS development and the contribution of specific lineages to ENS disease.

### SUMMARY

The disclosure relates to a method of differentiating at least one or a plurality of stem cells into at least one or a plurality of enteric neurons, the method comprising (i) exposing the one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons. In some embodiments, the method further comprises performing step (ii) after the neural crest cells are plated into one or a plurality of spheroids. In some embodiments, the differentiation factor is an amino acid sequence of BMP4 or a functional fragment thereof. In some embodiments, the differentiation factor is retinoic acid or an analogue thereof. In some embodiments, the differentiation factor is SB431542 or an analogue thereof. In some embodiments, the differentiation factor is an amino acid sequence of FGF2 or a functional fragment thereof. In some embodiments, the differentiation factor is CHIR 99021 or an analogue thereof.

In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (i) the one or plurality of stem cells are exposed to at least one or a combination of: BMP4 or a functional fragment thereof, SB431542 or an analogue thereof, and/or CHIR 99021 or an analogue thereof. In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (ii) the one or plurality of neural crest cells are exposed to at least one or a combination of: FGF2 or a functional fragment thereof, SB431542 or an analogue thereof, and/or CHIR 99021 or an analogue thereof, and retinoic acid or an analogue thereof.

The disclosure also provides a fully defined differentiation protocol that integrates retinoic acid (RA), effectively transitioning the induction of cranial NC to a specific vagal NC regional identity (16). In one aspect, a method of culturing pluripotent stem cells comprises:
(a) diluting pluripotent stem cells with a culture medium;
(b) centrifuging the pluripotent stem cell mixture to obtain a pellet and a supernatant;
(c) removing the supernatant from the pellet;
(d) adding culture medium to the pellet and resuspending the pluripotent stem cells in the culture medium;
(e) plating the resuspended pluripotent stem cells on a hydrogel disposed within a culture vessel; and
(f) incubating the pluripotent stem cells to a confluency of about 80%, wherein the culture medium.

In one aspect the culture medium is removed and replaced with fresh culture medium about every 2 days. Suitable culture medium includes E8-C medium. In some embodiments, the culture medium comprises a Rho kinase inhibitor, e.g., Y-27632. In some embodiments, the culture medium comprising the Rho-kinase inhibitor is removed from the culture vessel 3-5 hours after plating, followed by addition of E8-C medium free of any Rho kinase inhibitor to the culture vessel.

In one aspect, the pluripotent stem cells are human pluripotent stem cells, e.g., human ES cell line H9 (WA-09), human ES cell line UCSF4, and human iPS cell line WTC11.

In one aspect, the hydrogel comprises MATRIGEL® or vitronectin.

In one aspect, the pluripotent stem cells are passaged at least twice. In some embodiments, passaging comprises:
washing the pluripotent stem cells;
displacing the pluripotent stem cells by adding EDTA to the culture vessel; transferring the displaced pluripotent stem cells to a centrifuge tube;
centrifuging the to obtain a pellet;
adding culture medium to the centrifuge tube and resuspending the pluripotent stem cells in the pellet;
plating resuspended pluripotent stem cells; and
incubating the plated pluripotent stem cells to a confluency of about 80%, wherein the culture medium is removed and replaced about every other day.

In one embodiment, a method of producing an in vitro model of the enteric nervous system comprises:
i. contacting pluripotent stem cells to a first hydrogel disposed in a first culture vessel;
ii. applying a first culture medium into the first culture vessel in a volume sufficient to cover the pluripotent stem cells in contact with the first hydrogel;
iii. incubating the pluripotent stem cells for a first time and under conditions sufficient to grow a confluent layer of pluripotent stem cells;
iv. inducing the pluripotent stem cells for a second time and under conditions sufficient to differentiate the induced pluripotent stem cells into enteric neural crest cells;
v. transferring the neural crest cells to a second culture vessel;
vi. culturing the neural crest cells for a third time and under conditions for the neural crest cells to grow into enteric neural crest spheroids; and
vii. contacting the neural crest spheroids to a second hydrogel disposed in a third culture vessel;
viii. applying a second culture medium into the third culture vessel in a volume sufficient to cover the neural crest spheroids in contact with the second hydrogel; and
ix. incubating the neural crest spheroids for a third time and under conditions sufficient to differentiate the neural crest spheroids into enteric neurons;
   wherein the enteric neural crest cells comprise expression of about 1%, about 2%, about 3%, about 4%, about 5%, about 6%, about 7%, about 8%, about 9%, or about 10% CD49D and/or SOX10 higher than expressed by pluripotent stem cells;
   wherein the enteric neurons comprise expression of about 1%, about 2%, about 3%, about 4%, about 5%, about 6%, about 7%, about 8%, about 9%, or about 10% TUJ1 and TRKC higher than expressed by neural crest cells; and
   wherein the enteric neurons comprise less than about 20%, about 25%, about 30%, about 35%, about 40%, about 45%, about 50%, about 55%, about 60%, about 65%, about 70%, about 75% flat myofibroblast-like cells comprising expression of smooth muscle actin.

In one embodiment, the pluripotent stem cells are human ES cell line UCSF4, and wherein an induction efficiency at day 11 is at least 25%, at least 30%, or at least 35% as measured by expression of CD49D. In one embodiment, the induction efficiency at day 15 is at least 70%, at least 80%, or at least 90%.

In one embodiment, the pluripotent stem cells are human iPS cell line WTC11, and wherein an induction efficiency at day 11 is at least 10%, at least 15%, or at least 20% as measured by expression of CD49D. In one embodiment, the induction efficiency at day 15 is at least 65%, is at least 75%, or at least 85%. In one embodiment, the induction efficiency at day 20 is at least 25%, at least 30%, or at least 35% as measured by expression of TUJ1 and TRKC. In one embodiment, the induction efficiency at day 40 is at least 40%, at least 50%, or at least 60%. In one embodiment, the induction efficiency at day 55 is at least 50%, at least 55% or at least 60%.

In one embodiment, inducing the pluripotent stem cells for the second time and under conditions sufficient to differentiate the induced pluripotent stem cells into enteric neural crest cells (ENCs) comprises:
i. removing the first culture medium from the first culture vessel;
ii. adding a first ENC induction medium to the first culture vessel and incubating the differentiating pluripotent stem cells for two days;
iii. removing the first ENC induction medium from the first culture vessel;
iv. adding a second ENC induction medium to the first culture vessel and incubating the differentiating pluripotent stem cells for two days;
v. removing the second ENC induction medium;
vi. replacing the second ENC induction medium with fresh second ENC induction medium and incubating the differentiating pluripotent stem cells for two days;
vii. repeating steps v and vi;
viii. removing the second ENC induction medium;
ix. adding a third ENC induction medium and incubating the differentiating pluripotent stem cells for two days;
x. removing the third ENC induction medium;
xi. replacing the third ENC induction medium with fresh third ENC induction medium and incubating the differentiating pluripotent stem cells for two days; and
xii. obtaining enteric neural crest cells.

Suitable defined medium includes E8-C medium. In one embodiment, the first induction medium is free of a SMAD signaling inhibitor. In one embodiment, the first induction medium comprises BMP4. In one embodiment, the first induction medium is Cocktail A, as described in Example 1. In one embodiment, the second induction medium is Cocktail B, as described in Example 1. In one embodiment, the third induction medium comprises retinoic acid. In one embodiment, the third induction medium is Cocktail C, as described in Example 1.

Exemplary enteric neural crest cells express at least one of HoxB2, HoxB5, and PAX3 at about 1%, about 2%, about 3%, about 4%, about 5%, about 6%, about 7%, about 8%, about 9%, or about 10% higher than expressed by pluripotent stem cells.

In one embodiment, culturing the neural crest cells for the third time and under conditions for the neural crest cells to grow into enteric neural crest spheroids comprises incubating the neural crest cells in an ultra-low attachment culture vessel. In one embodiment, the third time is about 3 to about 4 days.

In one embodiment, the enteric neurons express at least one of CHAT, 5-HT, GABA, nNOS. In one embodiment, the CHAT induction efficiency is about 30% to about 50%. In one embodiment, the 5-HT induction efficiency is about 1% to about 15%. In one embodiment, the GABA induction efficiency is about 1% to about 20%. In one embodiment, the nNOS induction efficiency is about 1% to about 20%. In one embodiment, the enteric neurons comprise cholinergic and nitrergic neurons comprising co-expression of CHAT and NOS1 of at least about 1%, about 2%, about 3%, about 4%, about 5%, about 6%, about 7%, about 8%, about 9%, or about 10% greater than enteric neural crest cells. In one embodiment, enteric neurons comprise glial cells that express GFAP and SOX10 at least 5% greater than enteric neural crest cells.

In one embodiment, a system comprises:
a culture vessel comprising a hydrogel;
enteric neurons, wherein the enteric neurons are disposed in a two-dimensional layer on the hydrogel; and
a culture medium, wherein the culture medium is free of any SMAD signaling inhibitor,
wherein the enteric neurons are in culture for 5-20 days;
wherein the enteric neurons comprise less than about 20%, about 25%, about 30%, about 35%, about 40%, about 45%, about 50%, about 55%, about 60%, about 65%, about 70%, about 75% of cells comprising expression of smooth muscle actin.

In one embodiment, the cells comprising expression of smooth muscle actin are flat myofibroblast like cells and/or mesenchymal precursors.

In some embodiments, the culture vessel comprises a multi-well plate. In some embodiments, the hydrogel comprises MATRIGEL®, vitronectin, GELTREX®, and/or CULTREX® BME.

### BRIEF DESCRIPTION OF THE DRAWINGS

FIG. 1. Major subtypes of the embryonic NC along the anterior to-posterior axis. Migratory ENC progenitors are primarily derived from the vagal NC.

FIG. 2. Overview of protocol for deriving enteric neurons from hPSCs.

FIGS. 3A-3E. Induction of ENC cells from hPSCs. FIG. 3A Protocol (days 0-12) for ENC induction using option B. BMP4, Recombinant human bone morphogenetic protein-4; CHIR, CHIR 99021; RA, Retinoic Acid; SB, SB431542. FIG. 3B Confluency of hPSCs on day 0 of differentiation. FIG. 3C Phase contrast and SOX10::GFP reporter line GFP expression on day 2, day 6 and day 12. FIG. 3D Representative image of FACS analysis of CD49D/SOX10::GFP positive ENC cells on day 12. FIG. 3E Quantitative reverse transcriptase PCR (qRT-PCR) for vagal NC markers HOXB3, HOXB5, and ENC lineage marker PAX3 for ENC cells versus hPSCs. N=3 biological replicates. FC, fold change. Scale bars=200 μm.

FIGS. 4A and 4B. ENC spheroid culture. FIG. 4A. Protocol (days 12-15) for ENC spheroid formation. NB+N2+B27; NB/N2/B27, Neurobasal medium with N2 and B27 supplement; FGF2, Recombinant Human FGF Basic; CHIR, CHIR 99021. FIG. 4B. Phase contrast and SOX10::GFP reporter line GFP expression of 3D spheroids on day 14. Scale bar=200 μm.

FIG. 5. Induction of enteric neurons from ENC. Protocol for neuronal differentiation and maturation of ENC precursors. AA, ascorbic acid; GDNF, Recombinant Human Glial Derived Neurotrophic Factor.

FIGS. 6A-6F. Characterization of hPSC-derived ENC and enteric neurons. FIG. 6A. Flow cytometry analysis of CD49D positive ENC cells from hESC line UCSF4 and hiPSC line WTC11 on day 12. FIG. 6B. Flow cytometry analysis of CD49D positive ENC cells from hESC line UCSF4 and hiPSC line WTC11 after ENC spheroid enrichment on day 15. FIG. 6C. Immunofluorescence staining of TUJ1/TRKC on day 30 of EN induction. FIG. 6D. Flow cytometry analysis of TUJ1 and TRKC expression in EN cells on day 20, day 40 and day 55. FIG. 6E. Immunofluorescence images of CHAT, 5HT, NOS1, and GABA stained ENs on day 50. FIG. 6F. Flow cytometry analyses of CHAT, 5HT, NOS1, and GABA on ENS at day 75. AF647, Alexa Fluor™ 647. Scale bars=100 μm in c, f and 20 μm in e. o

FIGS. 7A and 7B. Expression of glial lineage markers hPSC-derived EN population. FIG. 7A. Immunofluorescence image of TUJ1/GFAP stained differentiated cultures on day 55. FIG. 7B. Flow cytometry analysis of SOX10 and GFAP expression on day 75 of differentiation. AF647, Alexa Fluor™ 647; AF488, Alexa Fluor™ 488.

FIGS. 8A-8E. Gene expression analysis of hPSC-derived enteric neurons. Quantitative reverse transcriptase PCR (qRT-PCR) of ENS lineage markers PHOX2B (FIG. 8A), EDNRB (FIG. 8B), ASCL1 (FIG. 8C), TUJ1 (FIG. 8D), CHAT (FIG. 8E), and GFAP (FIG. 8F) for EN populations versus hPSCs. N=3 biological replicates. FC, fold change.

FIGS. 9A and 9B. FACS purification of ENC lineages. Time course flow cytometry analysis of CD49D expression in unsorted differentiated cultures (FIG. 9A) and populations sorted at day 11 for CD49D (FIG. 9B). FSC, forward scatter; SSC, side scatter.

FIG. 10. Protocol (days 0-12) for ENC induction using option A. KSR, knockout serum replacement differentiation medium; LDN, LDN-193189, SB, SB431542, CHIR, CHIR 99021; RA, Retinoic Acid; SB, SB431542.

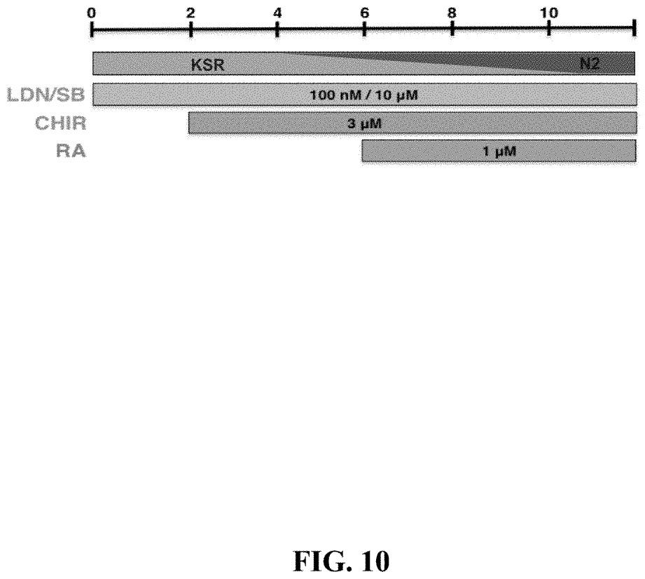

FIG. 11. Representative phase contrast image of WA09 embryonic stem cells cultured in E8 medium.

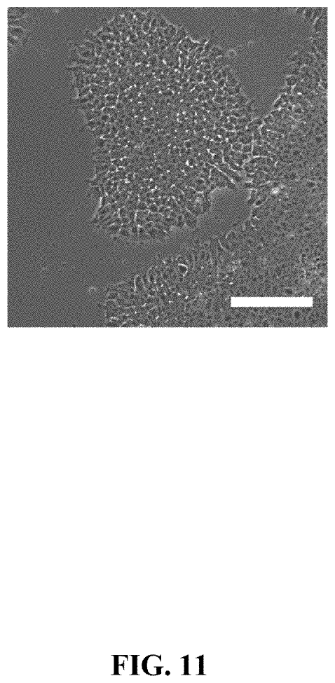

FIGS. 12A-12E. Representative phase contrast images of differentiating cells at different time points of EN induction.

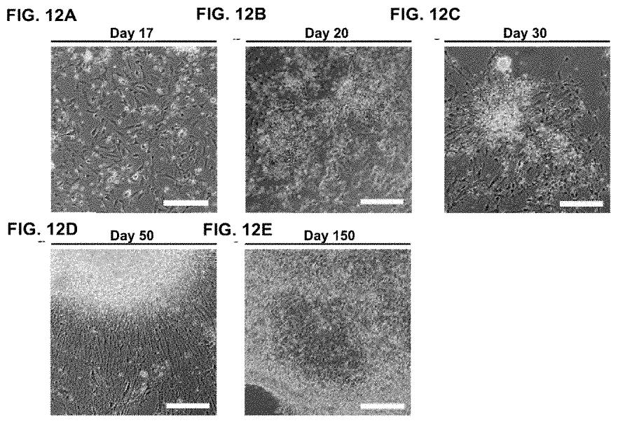

FIGS. 13A and 13B. Distinct populations of NOS1+ and CHAT+ cells in hESC-derived EN cultures. FIG. 13A. Immunofluorescence staining of NOS1 and CHAT on day 75 of EN induction. FIG. 13B. Flow cytometry analysis of NOS1 and CHAT expression on day 75 on EN induction. AF647, Alexa Fluor™ 647; AF488, Alexa Fluor™ 488.

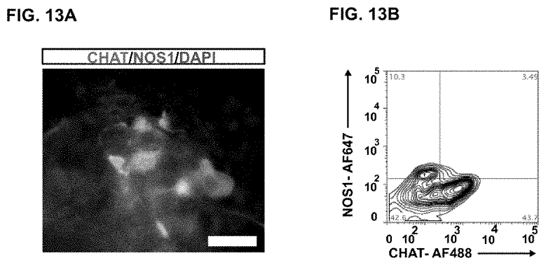

FIGS. 14A and 14B. Characterization of contaminating cells in hESC-derived EN cultures. FIG. 14A. Phase contrast image of low density regions of culture plates on day 75 of differentiation. Arrows point to flat non-neuronal contaminating cells. FIG. 14B. Immunofluorescence staining of EN cultures with SMA and TUJ1 on day 75 of differentiation.

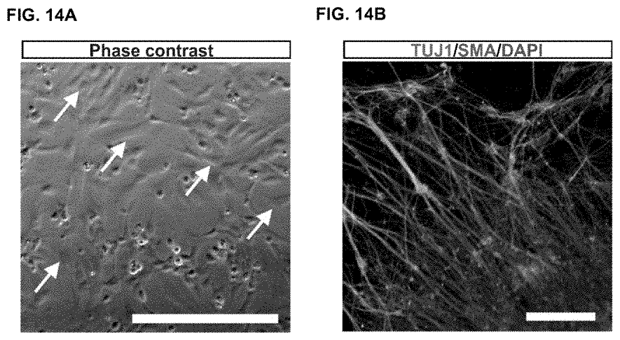

FIGS. 15A and 15B. Example of FACS gating strategy for purification of CD49D+ ENCs on day 12 of differentiation. FIG. 15A. Unstained control sample. FIG. 15B. Sample stained with CD49D.

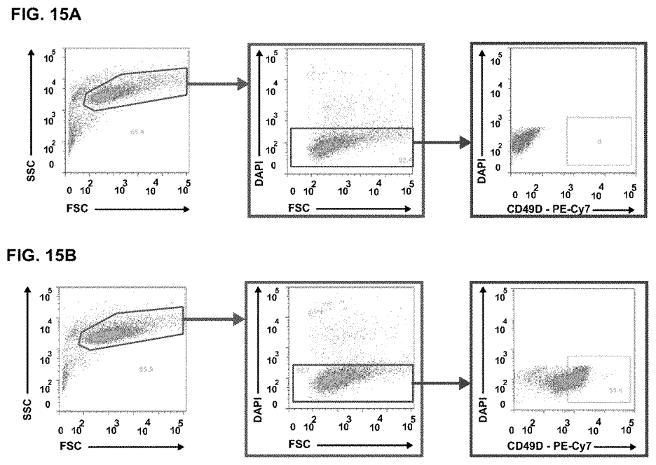

### DETAILED DESCRIPTION

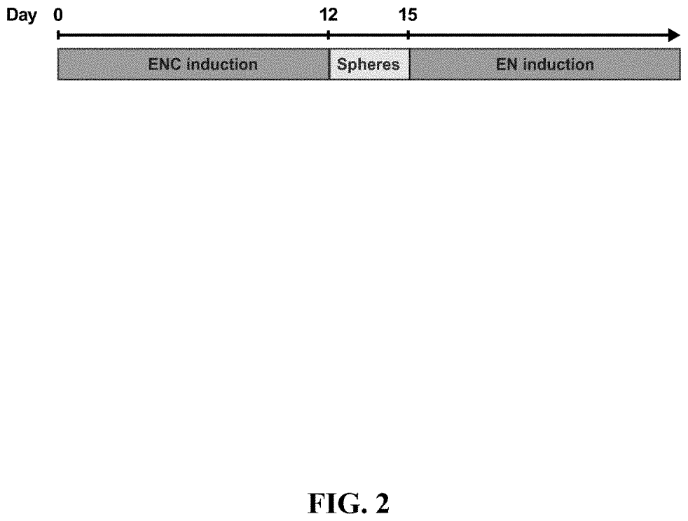

The disclosure provides novel protocols for derivation of enteric neurons from hPSCs (FIG. 2). It should be appreciated that such protocols find applications, for example, in probing the genetic contributions underpinning ENS pathogenesis using induced pluripotent stem cell (iPSC) lines generated from patients suffering from enteric neuropathies (20). Disease phenotypes can be modeled through in vitro differentiations and addressed via genetic or molecular perturbation strategies. Under the minimal, highly defined conditions of the disclosure, the inventors contemplate that the protocols of the disclosure will enable precise perturbations to observe the resulting cell fate commitments of EN progenitors, and/or to recapitulate disease phenotypes exhibited by EN lineages. The disclosure provides a scalable platform that produces unlimited numbers of hPSC-derived ENC cells or ENs on demand and enables high-throughput screening (HTS) assays that were previously unworkable. Therefore, the disclosure opens the door to testing the effects of large libraries of compounds or genes on fate commitments or the selective vulnerability of ENS lineages.

Further aspects of the disclosure include engrafting hPSC-derived ENC cells within host colons, e.g., murine host colon, and differentiate into functional ENs (16). Therefore, the inventors contemplate that EN cells of the disclosure find applications in regenerative medicine, e.g., to cure enteric neuropathies of the gastrointestinal tract via EN cell transplantation (21). The inventors contemplate use of the methods disclosed herein to derive ENs from hPSCs under highly defined conditions in the production clinical grade cells suitable for translational applications in the treatment of enteric neuropathies. The inventors further contemplate using the methods disclosed herein to produce pluripotent stem cell derived enteric neural cells of different cell type and state of differentiation. It should be appreciated that such cells may be used to replace damaged or absent cells relevant to enteric neuropathies. Moreover systems of the disclosure provide translational applications that present a rational approach for preclinical development and as research tools.

The protocol described herein provides improved methods for the derivation of enteric neural progenitors from pluripotent stem cells (22). Many labs in the stem cell field no longer rely on the support of feeder cells and have adopted the use of defined basal media, such as MTESR™ 1 (Stemcell Tech, 85850) or Essential 8 (Life Technologies, A2858501) for the maintenance of hPSC lines. Nevertheless, previous ENC induction methods commonly involve media containing serum replacement factors, namely knockout serum replacement (KSR), as is also the case in Comparative Example 2 (14, 20). In an effort to reduce the inconsistencies and quality control measures that undefined conditions may introduce to a protocol, we have pursued optimizing ENC induction in minimal, chemically defined conditions.

Recent studies have implemented alternative strategies for general NC induction using hPSCs, namely free floating embryoid body based approaches (23, 24). The migratory cells that come as a result of embryoid body and subsequent neural rosette formations have been shown to be positive for neural crest specific markers Sox10, TFAP2A, BRN3A, ISL1 and ASCL1, and a subset found to be positive for regionally specific vagal markers HOXB2 and HOXB5, even without the inclusion of RA (23). Overall neural crest induction efficiency was assessed by FACS of p75 and HNK1 double positive cells, a strategy used to isolate NC cells in previous protocols (Lee et al. 2007). Results showed >60% induction efficiency in ES cell line H9 and across independent hiPSC lines (23). Enriched NC populations were then co-cultured with primary gut explants in a Transwell system to promote ENC identities enriched for HOXB2, HOXB3, HAND2 and EDNRB. Notably, this method incorporates brain-derived neurotrophic factor (BDNF), glial cell line-derived neurotrophic factor (GDNF), nerve growth factor (NGF), neurotrophin-3 (NT3) into culture conditions. How these factors affect commitments of EN precursors, namely identities positive for VIP and calretinin (23), remains an interesting point of inquiry. A similar embryoid body approach incorporated brief exposure to RA during NC induction before eventually combining hPSC-derived NC cells with hPSC-derived intestinal organoids (HIOs) (24). In terms of the potential in ENC induction efficiency, comparative data between monolayer and embryoid body strategies remains limited. Indeed, the appropriate use of each strategy for a given application should be explored further.

The disclosure presents a protocol for the derivation of EN lineages from hPSCs. The development and utility of Comparative Example 2 was previously established in Fattahi et al., 2016 (16). The disclosure provides methods of deriving enteric neuron lineages following chemically defined and reliable methods.

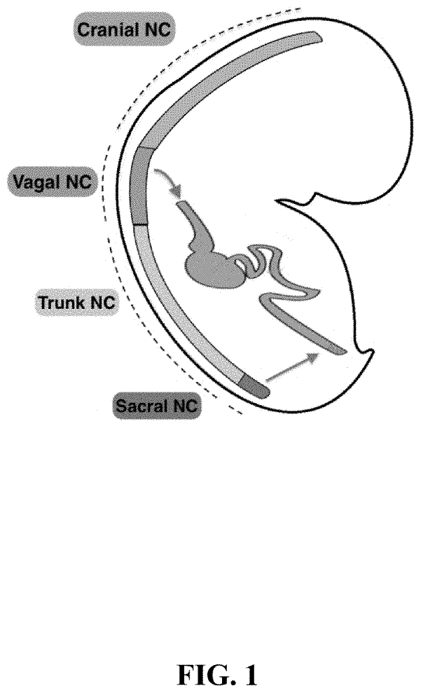

The important points of difference between Examples 1 and 2 are found in maintenance of hPSCs (Step 1) and during the ENC induction phase (Step 2). Adoption of Essential 8 (E8) offers a chemically defined basal media for the maintenance of hPSCs (26), in place of the feeder cell and KSR media used in Comparative Example 2. Transition from E8 to E6 basal media, in conjunction with precise combinations of BMP and Wnt signaling, and addition of RA, trigger the developmental cues required for ENC induction. Comparative Example 2 requires the gradual titration between relative amounts of basal media KSR and N2, while exemplary methods of the disclosure utilizes a single defined basal media E6. Consequently, Comparative example 1 involves dual SMAD inhibition using SB431542 and LDN-193189, while the conditions of the methods described herein only demand the TGFβ signaling inhibition using SB431542. As a result of replacing the KSR used in Comparative Example 2, early activation of low levels of BMP signaling with BMP4 induces NC specification under the defined conditions described herein. For both options, CHIR 99021 is used to activate canonical Wnt signaling, though lower concentrations are used in the conditions of the present disclose, and for both methods, retinoic acid is used to pattern NC cells towards the vagal ENC identity. A schematic illustration is provided outlining the induction conditions of Comparative Example 2 (Supplementary FIG. 1).

The disclosure relates to a method of differentiating at least one or a plurality of stem cells into at least one or a plurality of enteric neurons, the method comprising (i) exposing the one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons. In some embodiments, the method further comprises performing step (ii) after the neural crest cells are plated into one or a plurality of spheroids. In some embodiments, the differentiation factor is an amino acid sequence of BMP4 or a functional fragment thereof. In some embodiments, the differentiation factor is retinoic acid or an analogue thereof. In some embodiments, the differentiation factor is SB431542 or an analogue thereof. In some embodiments, the differentiation factor is an amino acid sequence of FGF2 or a functional fragment thereof. In some embodiments, the differentiation factor is CHIR 99021 or an analogue thereof.

In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (i) the one or plurality of stem cells are exposed to at least one or a combination of: BMP4 or a functional fragment thereof, SB431542 or an analogue thereof, and/or CHIR 99021 or an analogue thereof. In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (ii) the one or plurality of neural crest cells are exposed to at least one or a combination of: FGF2 or a functional fragment thereof, SB431542 or an analogue thereof, and/or CHIR 99021 or an analogue thereof, and retinoic acid or an analogue thereof. In some embodiments, the methods are free of steps of exposing any of the one or plurality of stem cells or neural crest cells to either of basal media KSR and N2 media.

In some embodiments the one or plurality of stem cells comprises an embryonic stem cell. In some embodiments, the one or plurality of stem cells comprises a pluripotent stem cell. In some embodiments the one or plurality of stem cells comprises a human embryonic stem cell. In some embodiments, the one or plurality of stem cells comprises a human pluripotent stem cell. In some embodiments, the one or plurality of stem cells comprises an induced human pluripotent stem cell. In some embodiments the one or plurality of stem cells comprises as hematopoetic stem cells, neural stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells.

Improved induction efficiency has been observed, when hPSCs are cultured under the maintenance conditions described in Examples 1 and 2 for several passages before differentiation. The density of hPSCs at the beginning of ENC induction also influences induction efficiency. In some embodiments the disclosure relates to a method of improving induction efficiency of stem cells into enteric neurons, the method comprising (i) exposing the one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons. In some embodiments, the method of improving induction efficiency of stem cells into enteric neurons comprises (i) exposing the one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons. In some embodiments, the method further comprises performing step (ii) after the neural crest cells are plated into one or a plurality of spheroids. In some embodiments, the differentiation factor is an amino acid sequence of BMP4 or a functional fragment thereof. In some embodiments, the differentiation factor is retinoic acid or an analogue thereof. In some embodiments, the differentiation factor is SB431542 or an analogue thereof. In some embodiments, the differentiation factor is an amino acid sequence of FGF2 or a functional fragment thereof. In some embodiments, the differentiation factor is CHIR 99021 or an analogue thereof. In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (i) the one or plurality of stem cells are exposed to at least one or a combination of: BMP4 or a functional fragment thereof, SB431542 or an analogue thereof, and/or CHIR 99021 or an analogue thereof. In some embodiments, the methods relate to i) exposing one or plurality of stem cells to a disclosed differentiation factor or a functional fragment thereof for a time period and in an amount sufficient to differentiate the one or plurality of stem cells into neural crest cells; and (ii) exposing the one or plurality of neural crest cells to a disclosed differentiation factor or a functional fragment thereof for a time period and in an amount sufficient to differentiate the one or plurality of neural crest cells into one or a plurality of enteric neurons; wherein in step (ii) the one or plurality of neural crest cells are exposed to at least one or a combination of: SB431542 or an analogue thereof, and/or CHIR 99021 or an analogue thereof, and retinoic acid or an analogue thereof.

In any of the disclosed methods, some embodiments are free of exposing any of the one or plurality of stem cells or neuronal crest cells to a SAMD inhibitor.

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, N.Y. 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” or “approximately” as used herein is meant to refer to within 5%, or more preferably within 1%, of a given value or range.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. in some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

The term “exposing” as used herein refers to bringing a disclosed compound and a cell, target receptor, or other biological entity together in such a manner that the compound can affect the activity of the cell (e.g., receptor, cell, etc.), either directly; i.e., by interacting with the target or cell itself, or indirectly; i.e., by interacting with another molecule, co-factor, factor, or protein on which the activity of the cell is dependent. In some embodiments, the activity of cell is differentiation. In some embodiments, the compound is one or more differentiation factors.

“Analogues” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pluripotent stem cell” as used herein is defined as a cell that is self-replicating capable of developing into cells and tissues of the three primary germ layers. Pluripotent stem cells include embryonic and induced pluripotent cells as defined herein. Contemplated pluripotent stem cells originate from mammals, e.g., human, mouse, rat, monkey, horse, goat, sheep, dog, cat etc.

The term “induced pluripotent stem cell” (iPSC) means a type of pluripotent cell made by reprogramming a somatic cell to have the same properties as embryonic stem cells, namely, the ability to self-renew and differentiate into the three primary germ layers. In some embodiments, iPSCs include mammalian cells, e.g., human, mouse, rat, monkey, horse, goat, sheep, dog, cat etc., reprogrammed to express Oct4, Nanog, Sox2, and optionally c-Myc. In some embodiments, iPSCs comprise reprogrammed primary cell lines. In some embodiments iPSCs are obtained from a repository, such as the Coriell Institute for Medical Research (e.g., Catalog ID GM25256 (WTC-11), GM25430, GM23392, GM23396, GM24666, GM27177, GM24683), California Institute for Regenerative Medicine: California's Stem Cell Agency (e.g., CW60261, CW60354, CW60359, CW60480, CW60335, CW60280, CW60594, CW60083, CW60086, CW60087 CW60167, CW60186), and the American Type Culture Collection (ATCC®) (e.g., ATCC-DYR0530 Human Induced Pluripotent Stem (IPS) Cells (ATCC® ACS-1012™, ATCC® ACS-1011™, ATCC® Number: ACS-1024™, ATCC® Number: ACS-1028™, ATCC® Number: ACS-1031™, ATCC® Number: ACS-1004™, ATCC® Number: ACS-1029™, ATCC® Number: ACS-1020™, ATCC® Number: ACS-1007™, ATCC® Number: ACS-1030™) Induced pluripotent stem cells may be derived from cell types such as fibroblasts taken from the skin, lung, or vein of subjects that are apparently healthy or diseased.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “embryonic stem cell line” as used herein is defined as a cell derived from the inner cell mass of the pre-implantation blastocyst capable of self-renewal and differentiation into the three primary germ layers. In some embodiments, embryonic stem cell lines listed in the NIH Human Embryonic Stem Cell Registry, e.g., CHB-1, CHB-2, CHB-3, CHB-4, CHB-5, CHB-6, CHB-8, CHB-9, CHB-10, CHB-11, CHB-12, RUES1, RUES2, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 26, HUES 27, HUES 28, CyT49, RUES3, WA01 (H1), UCSF4, NYUES1, NYUES2, NYUES3, NYUES4, NYUES5, NYUES6, NYUES7, MFS5, HUES 48, HUES 49, HUES 53, HUES 65, HUES 66, UCLA 1, UCLA 2, UCLA 3, WA07 (H7), WA09 (H9), WA13 (H13), WA14 (H14), HUES 62, HUES 63, HUES 64, CT1, CT2, CT3, CT4, MA135, Endeavour-2, WIBR1, WIBR2, HUES 45, Shef 3, Shef 6, WIBR3, WIBR4, WIBR5, WIBR6, BJNhem19, BJNhem20, SA001, SA002, UCLA 4, UCLA 5, UCLA 6, HUES PGD 13, HUES PGD 3, ESI-014, ESI-017, HUES PGD 11, HUES PGD 12, WA15, WA16, WA17, WA18, WA19, etc. In some embodiments, embryonic stem cells comprise gene(s) associated with diseases or disorders.

The term “enteric neural crest cell” means a cell produced by inducing differentiation of a pluripotent stem cell, wherein the enteric neural crest cell expresses SOX10, PHOX2B, EDNRB, TFAP2A, BRN3A, ISL1 and/or ASCL1. In some embodiments, the neural crest cell is present in an embryoid body or neural rosette. In some embodiments, the neural crest cell expresses vagal markers HOXB2, HOXB3, and/or HOXB5. In some embodiments, neural crest cells express p75 and HNK1. In some embodiments, neural crest cells express HOXB2, HOXB3, HAND2 and EDNRB.

The term “enteric neuron” means a cell produced by inducing differentiation of an enteric neural crest cell, wherein the enteric neuron exhibits downregulation of SOX10, sustained expression of EDNRB, ASCL1 and PHOX2B, and upregulation of TUJ1 and TRKC. In some embodiments enteric neurons express neuronal subtype specific markers including the cholinergic neuronal marker Choline Acetyl Transferase (CHAT), serotonin (5-HT) receptor, gamma-Aminobutyric acid (GABA), and neuronal nitric oxide synthase (nNOS). In some embodiments, CHAT expression indicates the presence of cholinergic neurons. In some embodiments, expression of NOS1 indicates the presence of nitrergic neurons. In some embodiments, enteric neurons include glial cells expressing glial fibrillary acidic protein (GFAP) and SOX10.

The term “rho kinase inhibitor” means a compound that decreases the activity of rho kinase. In some embodiments, the rho kinase inhibitor is N-[(3-Hydroxyphenyl)methyl]-N′-[4-(4-pyridinyl)-2-thiazolyl]urea dihydrochloride (RKI-1447), (+)-(R)-trans-4-(1-aminoethyl)-N-(4-pyridyl)cyclohexanecarboxamide dihydrochloride (Y-27632), Fasudil (HA-1077), Hydroxyfasudil (HA 1100 hydrochloride), Thiazovivin, GSK429286A, Narciclasine, and/or (+)-(R)-trans4-(1-aminoethyl)-N-(1H-pyrrolo[2,3-b]pyridin-4-yl)cyclohexanecarboxamide dihydrochloride (Y-30141).

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises MATRIGEL® or vitronectin. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “MATRIGEL®” means a solubilized basement membrane preparation extracted from the Engelbreth-Holm-Swarm (EHS) mouse sarcoma comprising ECM proteins including laminin, collagen IV, heparin sulfate proteoglycans, entactin/nidogen, and other growth factors. In some embodiments, CULTREX® BME (Trevigen, Inc.) or GELTREX® (Thermo-Fisher Inc.) may be substituted for MATRIGEL®.

The term “vitronectin” means a protein encoded by the VTN gene. In some embodiments, vitronectin has at least 70% sequence identity with SEQ ID NO: 1, SEQ ID NO: 2, SEQ ID NO: 3, or a fragment thereof.

>sp|P04004| VINC_HUMAN Vitronectin OS = Homo sapiens
OX = 9606 GN = VTN PE = 1 SV = 1
SEQ ID NO: 1
MAPLRPLLILALLAWVALADQESCKGRCTEGFNVDKKCQCDELCSYYQSCCTDYTAECKP
QVTRGDVFTMPEDEYTVYDDGEEKNNATVHEQVGGPSLTSDLQAQSKGNPEQTPVLKPEE
EAPAPEVGASKPEGIDSRPETLHPGRPQPPAEEELCSGKPFDAFTDLKNGSLFAFRGQYC
YELDEKAVRPGYPKLIRDVWGIEGPIDAAFTRINCQGKTYLFKGSQYWRFEDGVLDPDYP
RNISDGFDGIPDNVDAALALPAHSYSGRERVYFFKGKQYWEYQFQHQPSQEECEGSSLSA
VFEHFAMMQRDSWEDIFELLFWGRTSAGTRQPQFISRDWHGVPGQVDAAMAGRIYISGMA
PRPSLAKKQRFRHRNRKGYRSQRGHSRGRNQNSRRPSRATWLSLFSSEESNLGANNYDDY
RMDWLVPATCEPIQSVFFFSGDKYYRVNLRTRRVDTVDPPYPRSIAQYWLGCPAPGHL
>tr|Q3KR94| Q3KR94_RAT Vitronectin OS = Rattus norvegicus
OX = 10116 GN = Vtn PE = 1 SV = 1
SEQ ID NO: 2
MASLRPFFILALLALVSLADQESCKGRCTQGFMASKKCQCDELCTYYQSCCVDYMEQCKP
QVTRGDVFTMPEDEYWSYDYPEETKNSTSTGVQSENTSLHFNLKPRAEETIKPTTPDPQE
QSNTQEPEVGQQGVAPRPDTTDEGTSEFPEEELCSGKPFDAFTDLKNGSLFAFRGEYCYE
LDETAVRPGYPKLIQDVWGIEGPIDAAFTRINCQGKTYLFKGSQYWRFEDGVLDPDYPRN
ISEGFSGIPDNVDAALALPAHSYSGRERVYFFKGKQYWEYEFQQQPSQEECEGSSLSAVF
EHFALLQRDSWENIFELLFWGRSSDGAKGPQFISRDWHGVPGKVDAAMAGRIYITGSTFR
SVQAKKQKSGRRSRKRYRSRRGRGHSRSRSRSMSSRRPSRSVWFSLLSSEESGLGTYNYD
YDMNWRIPATCEPIQSVYFFSGDKYYRVNLRTRRVDSVNPPYPRSIAQYWLGCPTSEK
>sp|P29788| VINC_MOUSE Vitronectin OS = Mus musculus
OX = 10090 GN = Vtn PE = 1 SV = 2
SEQ ID NO: 3
MAPLRPFFILALVAWVSLADQESCKGRCTQGFMASKKCQCDELCTYYQSCCADYMEQCKP
QVTRGDVFTMPEDDYWSYDYVEEPKNNTNTGVQPENTSPPGDLNPRTDGTLKPTAFLDPE
EQPSTPAPKVEQQEEILRPDTTDQGTPEFPEEELCSGKPFDAFTDLKNGSLFAFRGQYCY
ELDETAVRPGYPKLIQDVWGIEGPIDAAFTRINCQGKTYLFKGSQYWRFEDGVLDPGYPR
NISEGFSGIPDNVDAAFALPAHRYSGRERVYFFKGKQYWEYEFQQQPSQEECEGSSLSAV
FEHFALLQRDSWENIFELLFWGRSSDGAREPQFISRNWHGVPGKVDAAMAGRIYVTGSLS
HSAQAKKQKSKRRSRKRYRSRRGRGHRRSQSSNSRRSSRSIWFSLFSSEESGLGTYNNYD
YDMDWLVPATCEPIQSVYFFSGDKYYRVNLRTRRVDSVNPPYPRSIAQYWLGCPTSEK

The term “biomarker” as used herein refers to a biological molecule present in an individual at varying concentrations useful in predicting the cancer status of an individual. A biomarker may include but is not limited to, nucleic acids, proteins and variants and fragments thereof. A biomarker may be DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest.

Choline Acetyl Transferase (CHAT) refers to an enzyme that catalyzes the transfer of an acetyl group from the coenzyme acetyl-CoA to choline, yielding acetylcholine (ACh). In some embodiments, CHAT has at least 70% sequence identity with SEQ ID NO: 4, SEQ ID NO: 5, SEQ ID NO: 6, or a fragment thereof.

>sp|P28329| CLAT_HUMAN Choline O-acetyltransferase OS = Homo
sapiens OX = 9606 GN = CHAT PE = 1 SV = 4
SEQ ID NO: 4
MGLRTAKKRGLGGGGKWKREEGGGTRGRREVRPACFLQSGGRGDPGDVGGPAGNPGCSPH
PRAATRPPPLPAHTPAHTPEWCGAASAEAAEPRRAGPHLCIPAPGLTKTPILEKVPRKMA
AKTPSSEESGLPKLPVPPLQQTLATYLQCMRHLVSEEQFRKSQAIVQQFGAPGGLGETLQ
QKLLERQEKTANWVSEYWLNDMYLNNRLALPVNSSPAVIFARQHFPGTDDQLRFAASLIS
GVLSYKALLDSHSIPTDCAKGQLSGQPLCMKQYYGLFSSYRLPGHTQDTLVAQNSSIMPE
PEHVIVACCNQFFVLDVVINFRRLSEGDLFTQLRKIVKMASNEDERLPPIGLLTSDGRSE
WAEARTVLVKDSTNRDSLDMIERCICLVCLDAPGGVELSDTHRALQLLHGGGYSKNGANR
WYDKSLQFVVGRDGTCGVVCEHSPFDGIVLVQCTEHLLKHVTQSSRKLIRADSVSELPAP
RRLRWKCSPEIQGHLASSAEKLQRIVKNLDFIVYKFDNYGKTFIKKQKCSPDAFIQVALQ
LAFYRLHRRLVPTYESASIRRFQEGRVDNIRSATPEALAFVRAVTDHKAAVPASEKLLLL
KDAIRAQTAYTVMAITGMAIDNHLLALRELARAMCKELPEMFMDETYLMSNRFVLSTSQV
PTTTEMFCCYGPVVPNGYGACYNPQPETILFCISSFHSCKETSSSKFAKAVEESLIDMRD
LCSLLPPTESKPLATKEKATRPSQGHQP
>sp|P32738| CLAT_RAT Choline O-acetyltransferase OS = Rattus
norvegicus OX = 10116 GN = Chat PE = 1 SV = 2
SEQ ID NO: 5
MPILEKAPQKMPVKASSWEELDLPKLPVPPLQQTLATYLQCMQHLVPEEQFRKSQAIVKR
FGAPGGLGETLQEKLLERQEKTANWVSEYWLNDMYLNNRLALPVNSSPAVIFARQHFQDT
NDQLRFAACLISGVLSYKTLLDSHSLPTDWAKGQLSGQPLCMKQYYRLFSSYRLPGHTQD
TLVAQKSSIMPEPEHVIVACCNQFFVLDVVINFRRLSEGDLFTQLRKIVKMASNEDERLP
PIGLLTSDGRSEWAKARTVLLKDSTNRDSLDMIERCICLVCLDGPGTGELSDTHRALQLL
HGGGCSLNGANRWYDKSLQFVVGRDGTCGVVCEHSPFDGIVLVQCTEHLLKHMMTSNKKL
VRADSVSELPAPRRLRLKCSPETQGHLASSAEKLQRIVKNLDFIVYKFDNYGKTFIKKQK
YSPDGFIQVALQLAYYRLYQRLVPTYESASIRRFQEGRVDNIRSATPEALAFVQAMTDHK
AAMPASEKLQLLQTAMQAHKQYTVMAITGMAIDNHLLALRELARDLCKEPPEMFMDETYL
MSNRFVLSTSQVPTTMEMFCCYGPVVPNGNGACYNPQPEAITFCISSFHSCKETSSVEFA
EAVGASLVDMRDLCSSRQPADSKPPAPKEKARGPSQAKQS
>sp|Q03059| CLAT_MOUSE Choline O-acetyltransferase OS = Mus
musculus OX = 10090 GN = Chat PE = 2 SV = 2
SEQ ID NO: 6
MPILEKVPPKMPVQASSCEEVLDLPKLPVPPLQQTLATYLQCMQHLVPEEQFRKSQAIVK
RFGAPGGLGETLQEKLLERQEKTANWVSEYWLNDMYLNNRLALPVNSSPAVIFARQHFQD
TNDQLRFAASLISGVLSYKALLDSQSIPTDWAKGQLSGQPLCMKQYYRLFSSYRLPGHTQ
DTLVAQKSSIMPEPEHVIVACCNQFFVLDVVINFRRLSEGDLFTQLRKIVKMASNEDERL
PPIGLLTSDGRSEWAKARTVLLKDSTNRDSLDMIERCICLVCLDGPGTGDLSDTHRALQL
LHGGGCSLNGANRWYDKSLQFVVGRDGTCGVVCEHSPFDGIVLVQCTEHLLKHMMTGNKK
LVRVDSVSELPAPRRLRWKCSPETQGHLASSAEKLQRIVKNLDFIVYKFDNYGKTFIKKQ
KCSPDGFIQVALQLAYYRLYQRLVPTYESASIRRFQEGRVDNIRSATPEALAFVQAMTDH
KAAVLASEKLQLLQRAIQAQTEYTVMAITGMAIDNHLLALRELARDLCKEPPEMFMDETY
LMSNRFILSTSQVPTTMEMFCCYGPVVPNGYGACYNPHAEAITFCISSFHGCKETSSVEF
AEAVGASLVDMRDLCSSRQPADSKPPTAKERARGPSQAKQS

“Serotonin receptors” or “5-hydroxytryptamine (5-HT) receptors” are G protein-coupled receptor and ligand-gated ion channels found in the central and peripheral nervous systems. Serotonin activates the serotonin receptors, mediating both excitatory and inhibitory neurotransmission. In some embodiments, serotonin receptors have at least 70% sequence identity with SEQ ID NO: 7, SEQ ID NO: 8, SEQ ID NO: 9, or a fragment thereof.

>sp|P08908| 5HT1A_HUMAN 5-hydroxytryptamine receptor 1A OS = Homo
sapiens OX = 9606 GN = HTR1A PE = 1 SV = 3
SEQ ID NO: 7
MDVLSPGQGNNTTSPPAPFETGGNTTGISDVTVSYQVITSLLLGTLIFCAVLGNACVVAA
IALERSLQNVANYLIGSLAVTDLMVSVLVLPMAALYQVLNKWTLGQVTCDLFIALDVLCC
TSSILHLCAIALDRYWAITDPIDYVNKRTPRRAAALISLTWLIGFLISIPPMLGWRTPED
RSDPDACTISKDHGYTIYSTFGAFYIPLLLMLVLYGRIFRAARFRIRKTVKKVEKTGADT
RHGASPAPQPKKSVNGESGSRNWRLGVESKAGGALCANGAVRQGDDGAALEVIEVHRVGN
SKEHLPLPSEAGPTPCAPASFERKNERNAEAKRKMALARERKTVKTLGIIMGTFILCWLP
FFIVALVLPFCESSCHMPTLLGAIINWLGYSNSLLNPVIYAYFNKDFQNAFKKIIKCKFC
RQ
>sp|P19327| 5HT1A_RAT 5-hydroxytryptamine receptor 1A OS = Rattus
norvegicus OX = 10116 GN = Htr1a PE = 1 SV = 1
SEQ ID NO: 8
MDVFSFGQGNNTTASQEPFGTGGNVTSISDVTFSYQVITSLLLGTLIFCAVLGNACVVAA
IALERSLQNVANYLIGSLAVTDLMVSVLVLPMAALYQVLNKWTLGQVTCDLFIALDVLCC
TSSILHLCAIALDRYWAITDPIDYVNKRTPRRAAALISLTWLIGFLISIPPMLGWRTPED
RSDPDACTISKDHGYTIYSTFGAFYIPLLLMLVLYGRIFRAARFRIRKTVRKVEKKGAGT
SLGTSSAPPPKKSLNGQPGSGDWRRCAENRAVGTPCTNGAVRQGDDEATLEVIEVHRVGN
SKEHLPLPSESGSNSYAPACLERKNERNAEAKRKMALARERKTVKTLGIIMGTFILCWLP
FFIVALVLPFCESSCHMPALLGAIINWLGYSNSLLNPVIYAYFNKDFQNAFKKIIKCKFC
RR
>sp|Q64264| 5HT1A_MOUSE 5-hydroxytryptamine receptor 1A OS = Mus
musculus OX = 10090 GN = Htr1a PE = 2 SV = 2
SEQ ID NO: 9
MDMFSLGQGNNTTTSLEPFGTGGNDTGLSNVTFSYQVITSLLLGTLIFCAVLGNACVVAA
TALERSLQNVANYLIGSLAVIDLMVSVLVLPMAALYQVLNKWILGQVICDLFIALDVLCC
TSSILHLCAIALDRYWAITDPIDYVNKRTPRRAAALISLTWLIGFLISIPPMLGWRTPED
RSNPNECTISKDHGYTIYSTFGAFYIPLLLMLVLYGRIFRAARFRIRKTVKKVEKKGAGT
SFGTSSAPPPKKSLNGQPGSGDCRRSAENRAVGTPCANGAVRQGEDDATLEVIEVHRVGN
SKGHLPLPSESGATSYVPACLERKNERTAEAKRKMALARERKTVKTLGIIMGTFILCWLP
FFIVALVLPFCESSCHMPELLGAIINWLGYSNSLLNPVIYAYFNKDFQNAFKKIIKCKFC

Gamma-Aminobutyric acid (GABA) acts as a trophic factor to modulate several essential developmental processes including neuronal proliferation, migration, and differentiation.

Neuronal nitric oxide synthase (nNOS) produces nitric oxide (NO) in the central and peripheral nervous systems. In some embodiments, nNOS has at least 70% sequence identity with SEQ ID NO: 10, SEQ ID NO: 11, SEQ ID NO: 12, or a fragment thereof.

>sp|P29475| NOS1_HUMAN Nitric oxide synthase, brain OS = Homo
sapiens OX = 9606 GN = NOS1 PE = 1 SV = 2
SEQ ID NO: 10
MEDHMFGVQQIQPNVISVRLFKRKVGGLGFLVKERVSKPPVIISDLIRGGAAEQSGLIQA
GDIILAVNGRPLVDLSYDSALEVLRGIASETHVVLILRGPEGFTTHLETTFTGDGTPKTI
RVTQPLGPPTKAVDLSHQPPAGKEQPLAVDGASGPGNGPQHAYDDGQEAGSLPHANGLAP
RPPGQDPAKKATRVSLQGRGENNELLKEIEPVLSLLTSGSRGVKGGAPAKAEMKDMGIQV
DRDLDGKSHKPLPLGVENDRVFNDLWGKGNVPVVLNNPYSEKEQPPTSGKQSPTKNGSPS
KCPRFLKVKNWETEVVLITTLHLKSTLETGCTEYICMGSIMHPSQHARRPEDVRTKGQLF
PLAKEFIDQYYSSIKRFGSKAHMERLEEVNKEIDTTSTYQLKDTELIYGAKHAWRNASRC
VGRIQWSKLQVFDARDCTTAHGMFNYICNHVKYATNKGNLRSAITIFPQRTDGKHDFRVW
NSQLIRYAGYKQPDGSTLGDPANVQFTEICIQQGWKPPRGRFDVLPLLLQANGNDPELFQ
IPPELVLEVPIRHPKFEWFKDLGLKWYGLPAVSNMLLEIGGLEFSACPFSGWYMGTEIGV
RDYCDNSRYNILEEVAKKMNLDMRKTSSLWKDQALVEINIAVLYSFQSDKVTIVDHHSAT
ESFIKHMENEYRCRGGCPADWVWIVPPMSGSITPVFHQEMLNYRLTPSFEYQPDPWNTHV
WKGINGTPTKRRAIGFKKLAEAVKFSAKLMGQAMAKRVKATILYATETGKSQAYAKTLCE
IFKHAFDAKVMSMEEYDIVHLEHETLVLVVISTFGNGDPPENGEKFGCALMEMRHPNSVQ
EERKSYKVRFNSVSSYSDSQKSSGDGPDLRDNFESAGPLANVRFSVFGLGSRAYPHFCAF
GHAVDTLLEELGGERILKMREGDELCGQEEAFRTWAKKVFKAACDVFCVGDDVNIEKANN
SLISNDRSWKRNKFRLTFVAEAPELTQGLSNVHKKRVSAARLLSRQNLQSPKSSRSTIFV
RLHTNGSQELQYQPGDHLGVFPGNHEDLVNALIERLEDAPPVNQMVKVELLEERNTALGV
ISNWTDELRLPPCTIFQAFKYYLDITTPPTPLQLQQFASLATSEKEKQRLLVLSKGLQEY
EEWKWGKNPTIVEVLEEFPSIQMPATLLLTQLSLLQPRYYSISSSPDMYPDEVHLTVAIV
SYRTRDGEGPIHHGVCSSWLNRIQADELVPCFVRGAPSFHLPRNPQVPCILVGPGTGIAP
FRSFWQQRQFDIQHKGMNPCPMVLVFGCRQSKIDHIYREETLQAKNKGVFRELYTAYSRE
PDKPKKYVQDILQEQLAESVYRALKEQGGHIYVCGDVTMAADVLKAIQRIMTQQGKLSAE
DAGVFISRMRDDNRYHEDIFGVTLRTYEVTNRLRSESIAFIEESKKDTDEVFSS
>sp|P29476| NOS1_RAT Nitric oxide synthase, brain OS = Rattus
norvegicus OX = 10116 GN = Nos1 PE = 1 SV = 1
SEQ ID NO: 11
MEENTFGVQQIQPNVISVRLFKRKVGGLGFLVKERVSKPPVIISDLIRGGAAEQSGLIQA
GDIILAVNDRPLVDLSYDSALEVLRGIASETHVVLILRGPEGFTTHLETTFTGDGTPKTI
RVTQPLGPPTKAVDLSHQPSASKDQSLAVDRVTGLGNGPQHAQGHGQGAGSVSQANGVAI
DPTMKSTKANLQDIGEHDELLKEIEPVLSILNSGSKATNRGGPAKAEMKDTGIQVDRDLD
GKSHKAPPLGGDNDRVFNDLWGKDNVPVILNNPYSEKEQSPTSGKQSPTKNGSPSRCPRF
LKVKNWETDVVLTDTLHLKSTLETGCTEHICMGSIMLPSQHTRKPEDVRTKDQLFPLAKE
FLDQYYSSIKRFGSKAHMDRLEEVNKEIESTSTYQLKDTELIYGAKHAWRNASRCVGRIQ
WSKLQVFDARDCTTAHGMFNYICNHVKYATNKGNLRSAITIFPQRTDGKHDFRVWNSQLI
RYAGYKQPDGSTLGDPANVQFTEICIQQGWKAPRGRFDVLPLLLQANGNDPELFQIPPEL
VLEVPIRHPKFDWFKDLGLKWYGLPAVSNMLLEIGGLEFSACPFSGWYMGTEIGVRDYCD
NSRYNILEEVAKKMDLDMRKTSSLWKDQALVEINIAVLYSFQSDKVTIVDHHSATESFIK
HMENEYRCRGGCPADWVWIVPPMSGSITPVFHQEMLNYRLTPSFEYQPDPWNTHVWKGTN
GTPTKRRAIGFKKLAEAVKFSAKLMGQAMAKRVKATILYATETGKSQAYAKTLCEIFKHA
FDAKAMSMEEYDIVHLEHEALVLVVTSTFGNGDPPENGEKFGCALMEMRHPNSVQEERKS
YKVRFNSVSSYSDSRKSSGDGPDLRDNFESTGPLANVRFSVFGLGSRAYPHFCAFGHAVD
TLLEELGGERILKMREGDELCGQEEAFRTWAKKVFKAACDVFCVGDDVNIEKPNNSLISN
DRSWKRNKFRLTYVAEAPDLTQGLSNVHKKRVSAARLLSRQNLQSPKFSRSTIFVRLHTN
GNQELQYQPGDHLGVFPGNHEDLVNALIERLEDAPPANHVVKVEMLEERNTALGVISNWK
DESRLPPCTIFQAFKYYLDITTPPTPLQLQQFASLATNEKEKQRLLVLSKGLQEYEEWKW
GKNPTMVEVLEEFPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVGPGTGIAPFRSFW
QQRQFDIQHKGMNPCPMVLVFGCRQSKIDHIYREETLQAKNKGVFRELYTAYSREPDRPK
KYVQDVLQEQLAESVYRALKEQGGHIYVCGDVTMAADVLKAIQRIMTQQGKLSEEDAGVF
ISRLRDDNRYHEDIFGVTLRTYEVTNRLRSESIAFIEESKKDADEVFSS
>sp|Q9Z0J4| NOS1_MOUSE Nitric oxide synthase, brain OS = Mus
musculus OX = 10090 GN = Nos1 PE = 1 SV = 1
SEQ ID NO: 12
MEEHTFGVQQIQPNVISVRLFKRKVGGLGFLVKERVSKPPVIISDLIRGGAAEQSGLIQA
GDIILAVNDRPLVDLSYDSALEVLRGIASETHVVLILRGPEGFTTHLETTFTGDGTPKTI
RVTQPLGTPTKAVDLSRQPSASKDQPLAVDRVPGPSNGPQHAQGRGQGAGSVSQANGVAI
DPTMKNTKANLQDSGEQDELLKEIEPVLSILTGGGKAVNRGGPAKAEMKDTGIQVDRDLD
GKLHKAPPLGGENDRVFNDLWGKGNVPVVLNNPYSENEQSPASGKQSPTKNGSPSRCPRF
LKVKNWETDVVLTDTLHLKSTLETGCTEQICMGSIMLPSHHIRKSEDVRTKDQLFPLAKE
FLDQYYSSIKRFGSKAHMDRLEEVNKEIESTSTYQLKDTELIYGAKHAWRNASRCVGRIQ
WSKLQVFDARDCTTAHGMFNYICNHVKYATNKGNLRSAITIFPQRTDGKHDFRVWNSQLI
RYAGYKQPDGSTLGDPANVEFTEICIQQGWKPPRGRFDVLPLLLQANGNDPELFQIPPEL
VLEVPIRHPKFDWFKDLGLKWYGLPAVSNMLLEIGGLEFSACPFSGWYMGTEIGVRDYCD
NSRYNILEEVAKKMDLDMRKTSSLWKDQALVEINIAVLYSFQSDKVTIVDHHSATESFIK
HMENEYRCRGGCPADWVWIVPPMSGSITPVFHQEMLNYRLTPSFEYQPDPWNTHVWKGTN
GTPTKRRAIGFKKLAEAVKFSAKLMGQAMAKRVKATILYATETGKSQAYAKTLCEIFKHA
FDAKAMSMEEYDIVHLEHEALVLVVTSTFGNGDPPENGEKFGCALMEMRHPNSVQEERKS
YKVRFNSVSSYSDSRKSSGDGPDLRDNFESTGPLANVRFSVFGLGSRAYPHFCAFGHAVD
TLLEELGGERILKMREGDELCGQEEAFRTWAKKVFKAACDVFCVGDDVNIEKANNSLISN
DRSWKRNKFRLTYVAEAPELTQGLSNVHKKRVSAARLLSRQNLQSPKSSRSTIFVRLHTN
GNQELQYQPGDHLGVFPGNHEDLVNALIERLEDAPPANHVVKVEMLEERNTALGVISNWK
DESRLPPCTIFQAFKYYLDITTPPTPLQLQQFASLATNEKEKQRLLVLSKGLQEYEEWKW
GKNPTMVEVLEEFPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVGPGTGIAPFRSFW
QQRQFDIQHKGMNPCPMVLVFGCRQSKIDHIYREETLQAKNKGVFRELYTAYSREPDRPK
KYVQDVLQEQLAESVYRALKEQGGHIYVCGDVTMAADVLKAIQRIMTQQGKLSEEDAGVF
ISRLRDDNRYHEDIFGVTLRTYEVTNRLRSESIAFIEESKKDTDEVFSS

Glial fibrillary acidic protein (GFAP) is a class-III intermediate filament. During the development of the central nervous system, GFAP is a cell-specific marker that distinguishes astrocytes from other glial cells. In some embodiments, GFAP has at least 70% sequence identity with SEQ ID NO: 13, SEQ ID NO: 14, SEQ ID NO: 15, or a fragment thereof.

>sp|P14136| GFAP_HUMAN Glial fibrillary acidic protein OS = Homo
sapiens OX = 9606 GN = GFAP PE = 1 SV = 1
SEQ ID NO: 13
MERRRITSAARRSYVSSGEMMVGGLAPGRRLGPGTRLSLARMPPPLPTRVDFSLAGALNA
GFKETRASERAEMMELNDRFASYIEKVRFLEQQNKALAAELNQLRAKEPTKLADVYQAEL
RELRLRLDQLTANSARLEVERDNLAQDLATVRQKLQDETNLRLEAENNLAAYRQEADEAT
LARLDLERKIESLEEEIRFLRKIHEEEVRELQEQLARQQVHVELDVAKPDLTAALKEIRT
QYEAMASSNMHEAEEWYRSKFADLTDAAARNAELLRQAKHEANDYRRQLQSLTCDLESLR
GTNESLERQMREQEERHVREAASYQEALARLEEEGQSLKDEMARHLQEYQDLLNVKLALD
IEIATYRKLLEGEENRITIPVQTFSNLQIRETSLDTKSVSEGHLKRNIVVKTVEMRDGEV
IKESKQEHKDVM
>sp|P47819| GFAP_RAT Glial fibrillary acidic protein OS = Rattus
norvegicus OX = 10116 GN = Gfap PE = 1 SV = 2
SEQ ID NO: 14
MERRRITSARRSYASSETMVRGHGPTRHLGTIPRLSLSRMTPPLPARVDFSLAGALNAGF
KETRASERAEMMELNDRFASYIEKVRFLEQQNKALAAELNQLRAKEPTKLADVYQAELRE
LRLRLDQLTTNSARLEVERDNLTQDLGTLRQKLQDETNLRLEAENNLAVYRQEADEATLA
RVDLERKVESLEEEIQFLRKIHEEEVRELQEQLAQQQVHVEMDVAKPDLTAALREIRTQY
EAVATSNMQETEEWYRSKFADLTDVASRNAELLRQAKHEANDYRRQLQALTCDLESLRGT
NESLERQMREQEERHARESASYQEALARLEEEGQSLKEEMARHLQEYQDLLNVKLALDIE
IATYRKLLEGEENRITIPVQTFSNLQIRETSLDTKSVSEGHLKRNIVVKTVEMRDGEVIK
ESKQEHKDVM
>sp|P03995| GFAP_MOUSE Glial fibrillary acidic protein OS = Mus
musculus OX = 10090 GN = Gfap PE = 1 SV = 4
SEQ ID NO: 15
MERRRITSARRSYASETVVRGLGPSRQLGTMPRFSLSRMTPPLPARVDFSLAGALNAGFK
ETRASERAEMMELNDRFASYIEKVRFLEQQNKALAAELNQLRAKEPTKLADVYQAELREL
RLRLDQLTANSARLEVERDNFAQDLGTLRQKLQDETNLRLEAENNLAAYRQEADEATLAR
VDLERKVESLEEEIQFLRKIYEEEVRELREQLAQQQVHVEMDVAKPDLTAALREIRTQYE
AVATSNMQETEEWYRSKFADLTDAASRNAELLRQAKHEANDYRRQLQALTCDLESLRGTN
ESLERQMREQEERHARESASYQEALARLEEEGQSLKEEMARHLQEYQDLLNVKLALDIEI
ATYRKLLEGEENRITIPVQTFSNLQIRETSLDTKSVSEGHLKRNIVVKTVEMRDGEVIKD
SKQEHKDVVM

Enteric neural crest cells express SOX10, which directs the activity of other genes that signal neural crest cells to become more specific cell types including enteric nerves. In some embodiments, SOX10 has at least 70% sequence identity with SEQ ID NO: 16, SEQ ID NO: 17, SEQ ID NO: 18, or a fragment thereof.

>sp|P56693| SOX10_HUMAN Transcription factor SOX-10 OS = Homo
sapiens OX = 9606 GN = SOX10 PE = 1 SV = 1
SEQ ID NO: 16
MAEEQDLSEVELSPVGSEEPRCLSPGSAPSLGPDGGGGGSGLRASPGPGELGKVKKEQQD
GEADDDKFPVCIREAVSQVLSGYDWTLVPMPVRVNGASKSKPHVKRPMNAFMVWAQAARR
KLADQYPHLHNAELSKTLGKLWRLLNESDKRPFIEEAERLRMQHKKDHPDYKYQPRRRKN
GKAAQGEAECPGGEAEQGGTAAIQAHYKSAHLDHRHPGEGSPMSDGNPEHPSGQSHGPPT
PPTTPKTELQSGKADPKRDGRSMGEGGKPHIDFGNVDIGEISHEVMSNMETFDVAELDQY
LPPNGHPGHVSSYSAAGYGLGSALAVASGHSAWISKPPGVALPTVSPPGVDAKAQVKTET
AGPQGPPHYTDQPSTSQIAYTSLSLPHYGSAFPSISRPQFDYSDHQPSGPYYGHSGQASG
LYSAFSYMGPSQRPLYTAISDPSPSGPQSHSP7HWEQPVYTTLSRP
>sp|O55170| SOX10_RAT Transcription factor SOX-10 OS = Rattus
norvegicus OX = 10116 GN = Sox10 PE = 1 SV = 1
SEQ ID NO: 17
MAEEQDLSEVELSPVGSEEPRCLSPSSAPSLGPDGGGGGSGLRASPGPGELGKVKKEQQD
GEADDDKFPVCIREAVSQVLSGYDWTLVPMPVRVNGASKSKPHVKRPMNAFMVWAQAARR
KLADQYPHLHNAELSKTLGKLWRLLNESDKRPFIEEAERLRMQHKKDHPDYKYQPRRRKN
GKAAQGEAECPGGETDQGGAAAIQAHYKSAHLDHRHPEEGSPMSDGNPEHPSGQSHGPPT
PPTTPKTELQSGKADPKRDGRSLGEGGKPHIDFGNVDIGEISHEVMSNMETFDVTELDQY
LPPNGHPGHVGSYSAAGYGLSSALAVASGHSAWISKPPGVALPTVSPPAVDAKAQVKTET
TGPQGPPHYTDQPSTSQIAYTSLSLPHYGSAFPSISRPQFDYSDHQPSGPYYGHAGQASG
LYSAFSYMGPSQRPLYTAISDPSPSGPQSHSPTHWEQPVYTTLSRP
>sp|Q04888| SOX10_MOUSE Transcription factor SOX-10 OS = Mus
musculus OX = 10090 GN = Sox10 PE = 1 SV = 2
SEQ ID NO: 18
MAEEQDLSEVELSPVGSEEPRCLSPGSAPSLGPDGGGGGSGLRASPGPGELGKVKKEQQD
GEADDDKFPVCIREAVSQVLSGYDWTLVPMPVRVNGASKSKPHVKRPMNAFMVWAQAARR
KLADQYPHLHNAELSKTLGKLWRLLNESDKRPFIEEAERLRMQHKKDHPDYKYQPRRRKN
GKAAQGEAECPGGEAEQGGAAAIQAHYKSAHLDHRHPEEGSPMSDGNPEHPSGQSHGPPT
PPTTPKTELQSGKADPKRDGRSLGEGGKPHIDFGNVDIGEISHEVMSNMETFDVTELDQY
LPPNGHPGHVGSYSAAGYGLGSALAVASGHSAWISKPPGVALPTVSPPGVDAKAQVKTET
TGPQGPPHYTDQPSTSQIAYTSLSLPHYGSAFPSISRPQFDYSDHQPSGPYYGHAGQASG
LYSAFSYMGPSQRPLYTAISDPSPSGPQSHSPTHWEQPVYTTLSRP

The term “two-dimensional culture” as used herein is defined as cultures of cells on flat hydrogels, including MATRIGEL® and vitronectin, disposed in culture vessels.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

A spheroid of the present invention can have any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 m, about 400 μm to about 500 μm, about 500 μm to about 1,000 μm, about 600 μm to about 1,000 μm, about 700 μm to about 1,000 μm, about 800 μm to about 1,000 am, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 m. In some embodiments, a spheroid may have a width, length, thickness, and/or diameter of about 50 μm, 100 μm, 200 μm, 300 μm, 400 μm, 500 am, 600 μm, 700 μm, 800 μm, 900 μm, 1,000 μm, 5,000 μm, 10,000 μm, 20,000 μm, 30,000 μm, 40,000 μm, or 50,000 m. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell.

In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types.

In some embodiments, the spheroids comprise at least two types of cells. In some embodiments the spheroids comprise neuronal cells and non-neuronal cells. In some embodiments, the spheroids comprise neuronal cells and astrocytes at a ratio of about 5:1, 4:1, 3:1, 2:1 or 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures.

The term “subject” as used herein refers to any animal (e.g., a mammal), including, but not limited to, humans, non-human primates, canines, felines, rodents, and the like. Preferably, the subject is a human subject. The terms “subject,” “individual,” and “patient” are used interchangeably herein. The terms “subject,” “individual,” and “patient” thus encompass individuals having cancer (e.g., breast cancer), including those who have undergone or are candidates for resection (surgery) to remove cancerous tissue.

A “therapeutically effective amount” of a therapeutic agent, or combinations thereof, is an amount sufficient to treat disease in a subject.

The terms “treating” or “treatment” or “treat” as used herein refer to therapeutic measures that cure, slow down, lessen symptoms of, and/or halt progression of a diagnosed pathologic condition or disorder.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

“Optional” or “optionally” means that the subsequently described event, circumstance, or material may or may not occur or be present, and that the description includes instances where the event, circumstance, or material occurs or is present and instances where it does not occur or is not present.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated stem cells and/or neural crest cells. In some embodiments, the culture vessel comprises enteric neurons. In some embodiments, the culture vessel comprises enteric neurons differentiated in culture form about 12 to about 20 days. In some embodiments, the culture vessel comprises a hydrogel and one or a plurality of isolated pluripotent stem cells.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. In some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments, the one or plurality of cells is stimulated by a differentiation factor. Differentiation factors may include one or a combination of any of the following:

### BMP4

MIPGNRMLMV VLLCQVLLGG ASHASLIPET
GKKKVAEIQG HAGGRRSGQS
HELLRDFEAT LLQMFGLRRR PQPSKSAVIP
DYMRDLYRLQ SGEEEEEQIH
STGLEYPERP ASRANTVRSF HHEEHLENIP
GTSENSAFRF LFNLSSIPEN
EVISSAELRL FREQVDQGPD WERGFHRINI
YEVMKPPAEV VPGHLITRLL
DTRLVHHNVT RWETFDVSPA VLRWTREKQP
NYGLAIEVTH LHQTRTHQGQ
HVRISRSLPQ GSGNWAQLRP LLVTFGHDGR
GHALTRRRRA KRSPKHHSQR
ARKKNKNCRR HSLYVDFSDV GWNDWIVAPP
GYQAFYCHGD CPFPLADHLN
STNHAIVQTL VNSVNSSIPK ACCVPTELSA
ISMLYLDEYD KVVLKNYQEM
VVEGCGCR

### FGF2

MVGVGGGDVE DVTPRPGGCQ ISGRGARGCN
GIPGAAAWEA ALPRRRPRRH
PSVNPRSRAA GSPRTRGRRT EERPSGSRLG
DRGRGRALPG GRLGGRGRGR
APERVGGRGR GRGTAAPRAA PAARGSRPGP
AGTMAAGSIT TLPALPEDGG
SGAFPPGHFK DPKRLYCKNG GFFLRIHPDG
RVDGVREKSD PHIKLQLQAE
ERGVVSIKGV CANRYLAMKE DGRLLASKCV
TDECFFFERL ESNNYNTYRS
RKYTSWYVAL KRTGQYKLGS KTGPGQKAIL
FLPMSAKS

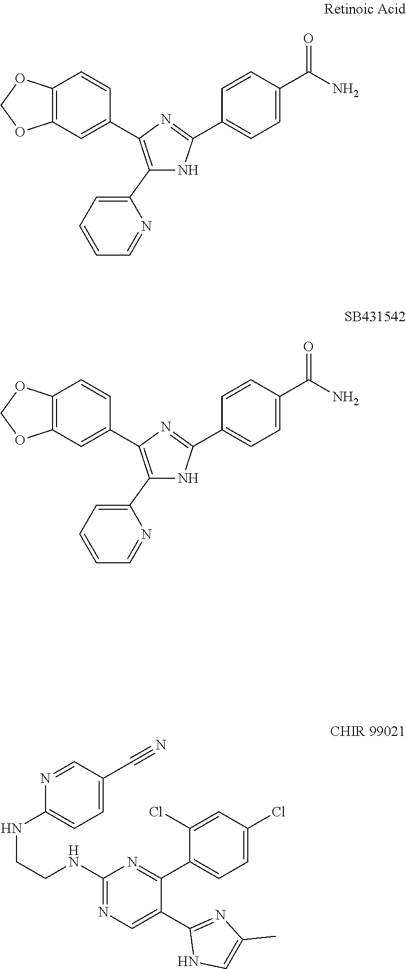 [SMILES: NC(=O)c1ccc(-c2nc(-c3ccc4c(c3)OCO4)c(-c3ccccn3)[nH]2)cc1 ; NC(=O)c1ccc(-c2nc(-c3ccc4c(c3)OCO4)c(-c3ccccn3)[nH]2)cc1 ; Cc1c[nH]c(-c2cnc(NCCNc3ccc(C#N)cn3)nc2-c2ccc(Cl)cc2Cl)n1]

In any of the methods or systems disclosed herein, the differentiation factors used may be functional fragments or variants of the polypeptides disclosed above with at least about 70% sequence identity to the above sequences. In any of the methods or systems disclosed herein, the differentiation factors used may be functional fragments or variants of the polypeptides disclosed above with at least about 80% sequence identity to the above sequences. In any of the methods or systems disclosed herein, the differentiation factors used may be functional fragments or variants of the polypeptides disclosed above with at least about 85% sequence identity to the above sequences. In any of the methods or systems disclosed herein, the differentiation factors used may be functional fragments or variants of the polypeptides disclosed above with at least about 90% sequence identity to the above sequences. In any of the methods or systems disclosed herein, the differentiation factors used may be functional fragments or variants of the polypeptides disclosed above with at least about 95% sequence identity to the above sequences. In any of the methods or systems disclosed herein, the differentiation factors used may be functional analogues of the small molecules disclosed above. The methods of the disclosure relate to the sequential exposure of a culture of cells to two or more different tissue culture mediums. In some embodiments, the methods relate to the sequential exposure of cells of the present disclosure to Cocktail Me

The present disclosure also relates to a system comprising: (i) a cell culture vessel optionally comprising a hydrogel; (ii) one or a plurality of stem cells or neural crest cells either in suspension or as a component of a spheroid; and (iii) on or plurality of differentiation factors. In some embodiments, the system further comprises one or combination of culture mediums disclosed herein. The disclosure also relates to a method of culturing enteric neurons in a system, the system comprising: (i) a cell culture vessel optionally comprising a hydrogel; (ii) one or a plurality of stem cells or neural crest cells either in suspension or as a component of a spheroid; and (iii) on or plurality of differentiation factors. In some embodiments, the system further comprises one or combination of culture mediums disclosed herein. In some embodiments, the methods relate to replacing medium during a culture time of form about 12 to about 21 days at least one time to (i) expose one or a plurality of stem cells to a first cell medium for a time period sufficient to differentiate the one or plurality of stem cells into neural crest cells and the sequentially replacing the medium to (ii) expose one or plurality of neural crest cells to a second cell medium for a time period sufficient to differentiate the one or plurality of neural crest cells into enteric neurons.

In some embodiments, the system comprises a solid substrate. The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2, 1 micron microns in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than 1 micron in diameter and comprises at least one layer of hydrogel matrix wherein the solid substrate comprises at least one compartment defined at least in part by the shape of an interior surface of the solid substrate and accessible from a point outside of the solid substrate by an opening, optionally positioned at one end of the solid substrate. In embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface. In some embodiments, the solid substrate and encourages directional growth of the cells away from the opening. In the case of neuronal cells, the degree of containment and shape of the at least one compartment are conducive to axon growth from soma positioned within the at least one compartment and at or proximate to the opening.

The present disclosure provides devices, methods, and systems involving production, maintenance, and physiological interrogation of neural cells in microengineered configurations designed to mimic native nerve tissue anatomy. It is another object of the disclosure to provide a medium to high-throughput assay of neurological function for the screening of pharmacological and/or toxicological properties of chemical and biological agents. In some embodiments, the agents are cells, such as any type of cell disclosed herein, or antibodies, such as antibodies that are used to treat clinical disease. In some embodiments, the agents are any drugs or agents that are used to treat human disease such that toxicities, effects or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease.

Similarly, information gathered from imaging can determine quantitative metrics for the degree of cell toxicology and lends further insight into toxic and neuroprotective mechanisms of various agents or compounds of interest. In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine, Vincristine, Vindesine, Vinorelbine, Vismodegib, and Vorinostat. In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin. In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate. In some embodiments, the at least one agent comprises one or a combination of anti-microbials chosen from: antibacterials, antifungals, antivirals, antiparasitics, heat, radiation, and ozone.

### EXAMPLES

Examples 1 and 2 were carried out with methods including, but not limited to, the following:

### Example 1. Defined Enteric Neuron Model System

Materials—Reagents and Equipment

E8-C, hPSC Medium for Maintenance

Combine Essential 8-Flex supplement (20 μl ml⁻¹) with ESSENTIAL 8™ Flex Medium. Store at 4° C. (use within 2 weeks).

Cocktail A, First ENC Differentiation Medium

Combine BMP4 (1 ng ml⁻¹), SB431542 (10 μM), CHIR 99021 (600 nM), with ESSENTIAL 6™ Medium. Store at 4° C. (use within 2 weeks).

Cocktail B, Second ENC Differentiation Medium

Combine SB431542 (10 μM), CHIR 99021 (1.5 μM), with ESSENTIAL 6™ medium. Store at 4° C. (use within 2 weeks).

Cocktail C, Third ENC Differentiation Medium

Combine SB431542 (10 μM), CHIR 99021 (1.5 μM), Retinoic Acid (1 μM), with ESSENTIAL 6™ medium. Store at 4° C. (use within 2 weeks).

NC-C, ENC Medium for Spheroid Maintenance

Combine FGF2 (10 ng ml⁻¹), CHIR 99021 (3 μM), N2 Supplement (10 μl ml⁻¹), B27 Supplement (20 μl ml⁻¹), Glutagro (10 μl ml⁻¹), MEM Nonessential Amino Acids (10 μl ml⁻¹), with NEUROBASAL® Medium. Store at 4° C. (use within 2 weeks).

EN-C, EN Medium for Differentiation and Maintenance

Combine GDNF (10 ng ml⁻¹), Ascorbic Acid (100 μM), N2 Supplement (10 μl ml⁻¹), B27 Supplement (20 μl ml⁻¹), Glutagro (10 μl ml⁻¹), MEM Nonessential Amino Acids (10 μl ml⁻¹), with NEUROBASAL® Medium. Store at 4° C. (use within 2 weeks).

EDTA 1× for Passaging hESCs

Combine EDTA (500 μM) with PBS.

MATRIGEL®

Thaw frozen vial of MATRIGEL® overnight at 4° C. Prepare 500 μl aliquots in pre-chilled 50 ml conical tubes using chilled pipette tips and keep frozen at −20° C.

MATRIGEL®-Coated Plates Dilute a 500 μl frozen aliquot of MATRIGEL® in 50 ml of cold DMEM:F12. Pipette up and down vigorously with a 25 ml or 50 ml serological pipette to break frozen Matrigel® pellet. Coat wells with the diluted MATRIGEL® solution (100 μl/cm² well surface area) and let stand in a 37° C. incubator overnight. Aspirate the MATRIGEL® solution before plating hPSCs.

Vitronectin-Coated Plates

Dilute vitronectin (10 μl ml⁻¹) with PBS and mix thoroughly. Coat wells with diluted vitronectin solution (100 μl/cm² well surface area) and let plates stand in a 37° C. incubator overnight. Aspirate the vitronectin solution before plating hPSCs. It should be appreciated that MATRIGEL®-coated plates yield a fully defined system, whereas vitronectin-coated plates yield a partially defined system.

PO/Lam/FN-Coated Plates

Combine PO (15 μg ml⁻¹) with PBS. Coat wells with PO/PBS solution (100 μl/cm² well surface area) and let stand in 37° C. incubator overnight. The following day, combine FN (2 μg ml⁻¹) and Laminin (2 μg ml⁻¹) with PBS. Aspirate PO/PBS and coat well with FN/LM/PBS solution (100 μl/cm² well surface area). Let plates stand in 37° C. incubator for a minimum of 2 hours. Aspirate FN/LM/PBS solution before plating cells.

Methods

Thawing Frozen hPSCs

Store frozen stocks of hPSCs in a liquid nitrogen cryogenic storage system at −156° C. For hPSCs lines that were previously maintained in mTESR1, first establish the line in mTESR1 for the initial passage, before transitioning the cultures to E8 medium. The cultures should be passaged at least twice in new medium before continuing the protocol.
1. Remove vial of hPSCs from liquid nitrogen and transfer vial to a 37° C. water bath.
2. Keep hold of the top of the sealed vial, and gently swirl around the water bath to ensure even thawing of frozen cells. Once only a small pellet of ice remains, remove the vial from water bath, spray the sealed vial with 70% ethanol, and transfer to laminar flow hood. Thawed cells should be plated immediately.
3. Add 0.5-1 ml of E8-C directly into vial and gently mix by pipetting up and down 1-2 times. Transfer cell suspension to a conical tube.
4. Centrifuge the conical tube at 1200 rpm (290×g) for 1 minute.
5. Carefully aspirate supernatant with a sterile pipette tip while avoiding contact with the pellet. Resuspend the pellet with 2 ml of E8-C and plate suspension into a single well of a 6-well or MATRIGEL®-coated or vitronectin-coated plate.
6. Proceed by expanding colonies as described in Step 1 of the protocol.
Note: A ROCK (Rho kinase) inhibitor such as Y-27632 dihydrochloride may be included in the initial E8-C medium conditions to enhance recovery and prevent excess cell death (27). Combine Y-27632 dihydrochloride (10 μM) with E8-C in a separate conical tube. Use this medium to break cell pellet after centrifugation and initial plating. Aspirate Y-27632 dihydrochloride supplemented medium from wells 3-5 hours after plating, and replace with fresh E8-C. Prolonged ROCK inhibition may adversely affect pluripotency and differentiation (28).

Step 1—Maintaining HPSC Cultures
7. Aspirate old E8-C medium from the corner of well using a sterile pipette tip. Add fresh E8-C (200 μl/cm² well surface area). Replace medium with fresh E8-C every other day.
8. When colonies are ˜80% confluent, begin passage by aspirating E8-C from the corner of a single well.
9. Add PBS (100 μl/cm² well surface area) and gently rock plate to wash off loose debris. Aspirate PBS using a sterile pipette tip.
10. Add EDTA 1×(100 μl/cm² well surface area). Replace lid of plate and watch for detachment of edges of colonies from well surface through an inverted microscope (2-4 minutes).
11. Use a P1000 micropipette or a 5 ml serological pipette to mechanically harvest colonies from the well. Transfer EDTA 1× cell suspension to a 15 ml conical tube.
Note: Pipetting too vigorously may lead to excessive colony dissociation and adversely affect cell viability. Total time in EDTA 1× and pipetting technique should be adjusted to maintain cell viability.
12. Centrifuge the conical tube at 1200 rpm (290×g) for 1 minute.
13. Carefully aspirate supernatant with a sterile pipette tip while avoiding contact with the pellet. Resuspend the pellet with E8-C and plate suspension in new Matrigel-coated or vitronectin-coated 6-well plate.
14. Label plate with cell line, date, and new passage number. Incubate at 5% CO₂ and 37° C.
Note: Passage hPSC cultures once every 5 days when they reach ˜80% confluency. For continued maintenance, passaging ratios generally vary between 1:12 and 1:18 (i.e., resuspend the pellet of cells collected from 1 well at ˜80% confluency with 2-3 ml of E8-C and transfer 1 ml of this suspension to a new 15 ml conical tube. Add fresh E8-C to the new tube to bring the total volume to 12 ml. Add 2 ml of this suspension to each well of a new 6 well plate).

Step 2-ENC Induction (Days 0-12)

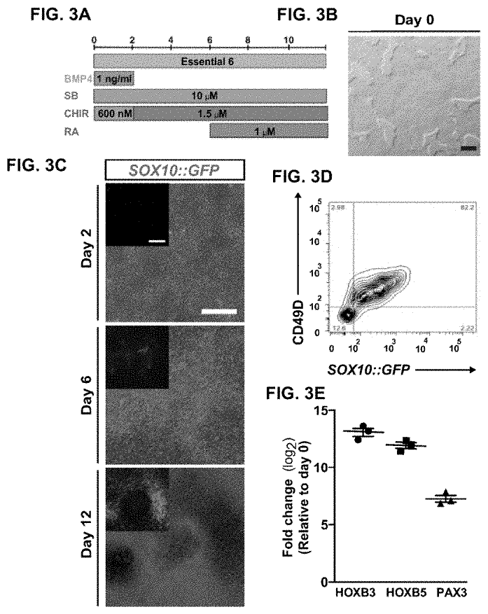

Day −2: Replating hPSCs for Differentiation
15. i. Two days before ENC induction, aspirate E8-C from hPSC cultures and use the same passage technique as described above, but use a 5:6 passaging ratio (i.e., all cells from 5 wells to a new 6-well plate) and leave in EDTA for 3-5 minutes for increased cell separation.
   ii. Feed cells with E8-C. Cells will continue to propagate and after 2 days the culture should become nearly confluent as a monolayer (FIG. 3b) while maintaining typical hPSC morphology (Supplementary FIG. 2).

Day 0: ENC Induction Begins
16. Aspirate old E8-C medium from corner of well using a sterile pipette tip. Add Cocktail A (200 μl/cm² well surface area). Record date of day 0 of ENC differentiation. Incubate at 5% CO₂ and 37° C.

Day 2
17. Aspirate Cocktail A from corner of well using a sterile pipette tip. Add Cocktail B (200 μl/cm² well surface area). Incubate at 5% CO₂ and 37° C.

Day 4
18. On day 4, aspirate old Cocktail B using a sterile pipette tip and add fresh Cocktail B (200 μl/cm² well surface area). Incubate at 5% CO₂ and 37° C.

Day 6
19. On day 6, aspirate Cocktail B using a sterile pipette tip. Add Cocktail C (400 μl/cm² well surface area). Incubate at 5% CO₂ and 37° C. At ˜day 6, SOX10::GFP⁺ cells begin to cluster within the monolayer, indicating SOX10⁺ ENC lineage identity. GFP⁺ cluster size and prevalence continue to increase over the remaining ENC differentiation (FIG. 3c).

Day 8
20. On day 8, aspirate old Cocktail C using a sterile pipette tip and add fresh Cocktail C (400 μl/cm² well surface area). Incubate at 5% CO₂ and 37° C.
Note: As confluency continues to increase over the course of NC induction, cells may detach from the underlying monolayer. Avoid excess loss of cells by tipping the plate and gently adding fresh media to corner and side of well.

Day 10
21. On day 10, aspirate old Cocktail C using a sterile pipette tip and add fresh Cocktail C, increasing volume to 600 μl/cm² of well surface area. Incubate at 5% CO₂ and 37° C.

Day 11/12
22. ENC cells are ready to be removed for further differentiation. ENC cells are characterized by co-expression of SOX10::GFP and CD49D (FIG. 3d). ENC lineages are confirmed by the expression of HoxB2, HoxB5, and PAX3 (FIG. 3e). Optional purification of ENC populations can be prepared by FACS using CD49D surface marker staining.
Note: Transfer ENC differentiations on day 11 if SOX10::GFP+ clusters are detaching from monolayer. Otherwise, day 12 will mark a complete ENC induction period.

Step 3-ENC Spheroid (Day 12-15)

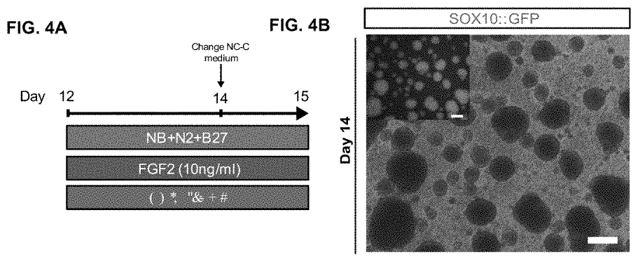

ENC monolayers are detached from the well surface and transferred to ultra-low attachment plates to form free floating 3D spheroids. Spheroids are maintained in NC-C medium for 3-4 days as part of a NC maintenance process (FIG. 4A).
23. On day 11- to 12, aspirate Cocktail C from ENC induction phase plate using a sterile pipette tip. Add Accutase (100 μl/cm² well surface area). Incubate for 30 minutes at 37° C. and 5% CO₂.
24. Without aspirating Accutase, add NC-C (100 μl/cm² well surface area). Use a serological pipette to mechanically harvest cells from the surface of well. Add the cell suspension to a 15 ml conical tube.
25. Centrifuge the conical tube at 1200 rpm (290×g) for 1 minute.
26. With a sterile pipette tip, carefully aspirate as much supernatant as possible while avoiding the cell pellet.
27. Resuspend the pellet with the appropriate volume of NC-C and transfer the cell suspension to an ultra-low attachment 6-well plate (2 ml/well). 10 cm² of ENC monolayer will be transferred to 1 well of an ultra-low attachment 6 well plate (i.e. A 6-well ENC induction plate corresponds to a 6 well ultra-low attachment plate). Incubate at 37° C. and 5% CO₂.
28. On day 14, gently swirl ultra-low attachment plates to group the free-floating spheroids into the center of each well. Using a P1000 micropipette, slowly aspirate the old NC-C by moving around the circumference of well, actively avoiding any removal of spheroids.
29. Add 2 ml of fresh NC-C to each ultra-low attachment plate well. Incubate at 37° C. and 5% CO₂. 3D spheroids should form by day 14 (FIG. 4b).

Step 4-EN Induction Phase (Day 15-)

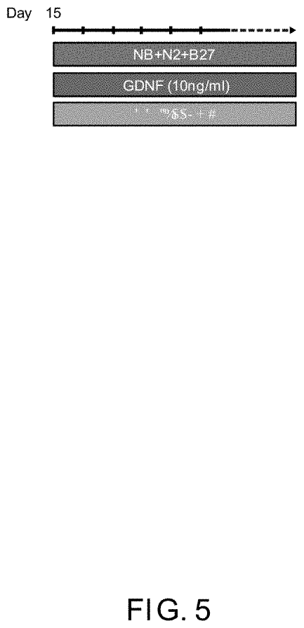

After the ENC spheroid phase (Step 3) and 15 total days from the start of ENC differentiation, ENC spheroids are dissociated with Accutase treatment and replated on PO/LM/FN-coated wells. This step marks the final replating of the protocol and the beginning of EN induction (FIG. 5).
30. On day 15, gently swirl ultra-low attachment plates to group the free-floating spheroids into center of well. Using a P1000 micropipette, slowly remove the old NC-C from the circumference of well while actively avoiding any removal of spheroids.
31. Add Accutase (1 ml) to each well and incubate for 30 minutes at 37° C. and 5% CO₂.
32. Use a 5 ml serological pipette to gently dissociate the remaining spheroids by 2-3 rounds of pipetting. Transfer the cell suspension to a 50 ml conical tube.
Note: Dissociation of spheroids using a P1000 micropipette adds an element of shear stress and may lead to excessive cell death. The use a serological pipette is recommended due to the larger diameter of the tip opening.
33. Centrifuge the conical tube at 1200 rpm (290×g) for 1 minute.
34. Carefully aspirate supernatant using a sterile pipette tip while avoiding contact with the cell pellet.
35. Resuspend the pellet in 10 ml of EN-C.
36. Determine the viable cell concentration using a hemocytometer and Trypan Blue.
37. Add the remaining volume of EN-C to replate the cell suspension at ˜100,000 cells/cm² of surface area to the conical tube.
38. Aspirate the FN/Laminin/PBS solution from wells using a sterile pipette tip.
39. Add the EN-C cell suspension to center of the well or dish.
40. Incubate at 37° C. and 5% CO₂. Move EN plates in a north/south/east/west direction upon returning to incubator shelf to insure even distribution of cell attachment.
41. Replace EN-C medium (200 μl/cm² well surface area) every other day until 30- to 40-days after the start of ENC induction.
Note: After 30- to 40-days of differentiation, reduce EN-C medium replacement to 1- to 2-times a week but increase volume to 400 μl/cm². If cultures begin detaching from the surface of the well, supplement EN-C with FN (2 μg ml⁻¹) and LM (2 μg ml⁻¹).

Results

The disclosed methods and systems reliably produce populations of hPSC-derived ENs under chemically defined conditions. Proportions of cells positive for EN identities may vary between cell lines, as well as between differentiations of a given cell line. Regardless, cells possessing a neuronal morphology should emerge by 20 days after the start of hPSC differentiation (Supplementary FIGS. 3A and 3B) and stay viable for several weeks (Supplementary FIGS. 3C and 3E). Neuronal identity is confirmed through marker expression and relative gene expression analysis by qRT-PCR.

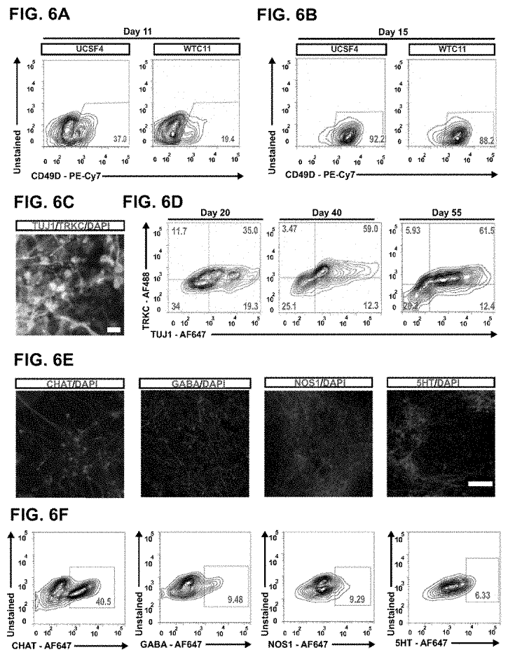

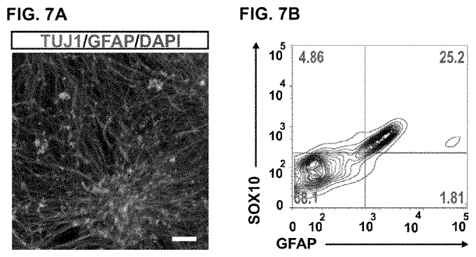

The identification of CD49D (α4 integrin) as a reliable surface marker of SOX10+NC lineages (16), enables the assessment of the ENC induction efficiency and their prospective isolation. Analysis of CD49D expression after 12 and 15 days of differentiation under the disclosed method for two additional hPSC lines (hESC-UCSF4 and hiPSC-WTC11) (FIGS. 6A and 6B) demonstrated initial variation in ENC induction efficiency between cell lines and validated the ENC spheroid phase (day 12-day 15) as for the enrichment of CD49D+ enteric neuron precursors (FIG. 6B). After EN induction, neuronal identity is verified based on co-expression of pan-neuronal marker TUJ1 and enteric neuron precursor specific marker TRKC (FIGS. 6C and 6D). Expression of additional neuronal subtype specific markers include the cholinergic neuronal marker Choline Acetyl Transferase (CHAT), serotonin (5-HT), gamma-Aminobutyric acid (GABA) and neuronal nitric oxide synthase (nNOS) which labels nitric oxide (NO) producing neurons (FIGS. 6E and 6F). Co-expression analysis of CHAT and NOS1 reveals separate population of cholinergic and nitrergic neurons in the differentiated culture (Supplementary FIG. 4). Glial cells expressing glial fibrillary acidic protein (GFAP) and SOX10, also emerge in differentiated cultures at the later stages of EN induction step (FIGS. 7A and 7B).

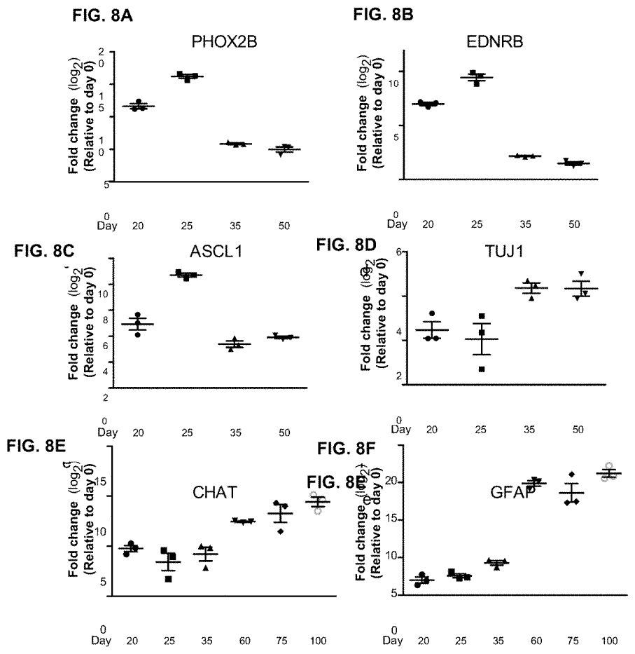

Comparisons of relative gene expression between samples collected from separate time-points during differentiation reveal population level transitions in gene expression that are supported by previous descriptions of the transcriptional processes of in vivo ENS development (29). High expression levels of ENC-derived progenitor markers PHOX2B, ASCL1, and EDNRB during the transition to EN induction reveal the presence of enteric precursors (FIGS. 8A-8C). The synchronous downregulation of precursor markers with upregulation of TUJ1 and CHAT illustrates neuronal commitments and maturity taking place over the course of EN induction (FIGS. 8D and 8E). Additionally, the delayed emergence of enteric glia is seen by the increased expression of glial marker GFAP in the later stages of EN induction phase (FIG. 8F).

NC-derived flat myofibroblast-like cells identifiable by expression of smooth muscle actin (SMA) have also been observed (Supplementary FIG. 5). These SMA-expressing cells catalyze the detachment of neurons from the well surface and apoptosis. Minimizing the number cells expressing SMA has been associated with improving the overall durability of enteric neuron populations.

### Example 2. Comparative Example of a Partially Defined Enteric Neuron Model System

Materials—Reagents and Equipment

ES Medium, hPSC Medium for Maintenance

Combine 100 ml of KSR to 400 ml DMEM/F12, no glutamine. Add 5 ml of 200 mM L-glutamine, and 5 ml of MEM Nonessential Amino Acids. Filter sterilize, then add 10 ng/ml of recombinant FGF2. Store at 4° C. (use within 2 weeks).

MEF Medium, MEF Culture Medium

Combine 100 ml FBS to 900 ml of DMEM. Filter sterilize before use. Store at 4° C. (use within 3 weeks).

KSR Medium, Early ENC Differentiation Medium

Combine 410 ml of Knockout DMEM, 75 ml of KSR, 5 ml of 200 mM L-glutamine), 5 ml of MEM non-essential amino acids, and 500 μl of 2-mercaptoethanol. Store at 4° C. (use within 3 weeks).

N2 Medium, Late ENC Differentiation Medium

Dissolve one bag of DMEM/F12 powder in 550 ml of distilled water. Add: 1.55 g of glucose, 2.00 g of sodium bicarbonate, 16.1 μg putrescine, 32 μg progesterone, 5.2 μg sodium selenite, 100 mg transferrin, 25 mg insulin (dissolved in 10 ml of 5 mM NaOH). Add double-distilled water (with a resistance of 18.2 M) to a final volume of 1000 ml. Filter sterilize and store at 4° C. in Option A (use within 3 weeks).

MEF-Coated Dishes

Prepare MEF coated 10-cm dish at least one day before hPSC passaging by coating culture surface with 0.1% gelatin dissolved in PBS (5 ml). Incubate at room temperature for 10 minutes. Thaw vial of mitomycin-C treated MEFs in a 37° C. water bath and resuspend cells in MEF medium (100,000 cells ml⁻¹). Aspirate 0.1% gelatin and add ˜1.2×10⁶ MEFs to 10-cm dish (15,000 cells/cm² well surface area). Culture MEFs overnight in a 37° C. incubator. MEF coated dishes may be left cultured for up to 3 days before plating hPSCs.

Methods

Thawing Frozen hPSCs

Store frozen stocks of hPSCs in a liquid nitrogen cryogenic storage system at −156° C. For hPSCs lines that were previously maintained in mTESR1, first establish the line in mTESR1 for the initial passage, before transitioning the cultures to KSR based hES medium. The cultures should be passaged at least twice in new medium before continuing the protocol.

Plating hPSCs is performed as described in Example 1, substituting hESC-medium for E8-C medium and 6-well MEF-coated plates for MATRIGEL®-coated or vitronectin-coated plates.

Step 1—Maintaining HPSC Cultures
1. On the day of passaging, aspirate human ES cell medium from hPSC culture and add PBS (10 ml/10-cm dish). Gently rock the dish to wash cultures and aspirate off PBS.
2. Add collagenase IV (2 ml/10-cm dish) and incubate at room temperature for 10 min.
3. Aspirate collagenase IV and add PBS (10 ml/10-cm dish). Gently rock the dish to wash colonies and aspirate off PBS.
4. Use a cell scraper to displace colonies from the culture surface.
5. Resuspend detached colonies in 1 ml of human ES cell medium and pipet up and down to disassociate larger colonies.
6. Add appropriate volume of colony suspension with enough human ES cell medium for replating.
7. Aspirate MEF medium from cultured MEF dish and add ES cell suspension.
8. Label plate with cell line, date, and new passage number. Incubate at 5% CO₂ and 37° C.
Note: Passage hPSC cultures once a week when they reach ˜80% confluency. For continued maintenance, passaging ratios generally vary between 1:6 and 1:12 (i.e., resuspend the pellet of cells collected from 1 well at ˜80% confluency with 12 ml of fresh hESC medium. Add 2 ml of this suspension to each well of a new 6 well plate).

Step 2-ENC Induction (Days 0-12)

Day −1: Replating hPSCs for differentiation
9. i. On the day before the start of ENC induction, remove human ES cell medium from hPSC colonies and add PBS (10 ml/10-cm dish). Replace plate lid and gently rock the dish to wash colonies and aspirate the PBS.
   ii. Add 0.05% trypsin (2 ml/10-cm dish) and vigorously shake back and forth for 1 to 2 minutes to detach MEFs. MEFs should detach before hPSC colonies. Aspirate medium containing MEFs, leaving hPSC colonies attached. Let dish stand without medium for 1 minute at room temperature.
   iii. Add human ES cell medium supplemented with Y-27632 (10 μM) and mechanically detach colonies by pipetting up and down using a P1000 pipet. As Dissociate the cells more than during hPSC maintenance passaging to separate the cells into single cells or small clusters of 5-10 cells.
   iv. Aspirate Matrigel coating solution from coated plates and add fresh human ES cell medium supplemented with Y-27632. Plate ˜100,000 cells/cm² onto Matrigel coated plates containing human ES cell medium supplemented with Y-27632. Incubate overnight at 37° C. and 5% CO₂.

Day 0: Neural Crest Induction Begins
10. When monolayer is ˜70% confluent, aspirate human ES cell medium from dish and add fresh KSR medium supplemented with SB431542 (10 μM) and LDN-193189 (1 μM).

Day 2
11. Aspirate old medium and add fresh KSR medium supplemented with SB431542 (10 μM), LDN-193189 (1 μM), and CHIR-99021 (3 μM).

Day 4
12. Aspirate old medium and add a mixture of 75% KSR and 25% N2 medium supplemented with SB431542 (10 μM), LDN-193189 (1 μM), and CHIR-99021 (3 μM).

Day 6
13. Aspirate old medium and add a mixture of 50% KSR and 50% N2 medium supplemented with SB431542 (10 μM), LDN-193189 (1 μM), CHIR-99021 (3 μM), and Retinoic Acid (1 μM).

Day 8
14. Aspirate old medium and add a mixture of 25% KSR and 75% N2 medium supplemented with SB431542 (10 μM), LDN-193189 (1 μM), CHIR-99021 (3 μM), and Retinoic Acid (1 μM).

Day 10
15. Aspirate old medium and add N2 medium supplemented with SB431542 (10 μM), LDN-193189 (1 μM), CHIR-99021 (3 μM), and Retinoic Acid (1 μM).

Day 11/12
22. ENC cells are ready to be assayed or further differentiated.
Note: As confluency continues to increase over the course of NC induction, cells may detach from the underlying monolayer. Avoid excess loss of cells by tipping the plate and gently adding fresh media to corner and side of well. Please refer to Fattahi et. al., 2015 (13) for representative images of differentiated culture at various time point during differentiation.

Step 3-ENC Spheroid (Day 12-15)

ENC monolayers are detached from the well surface and transferred to ultra-low attachment plates to form free floating 3D spheroids as described in Example 1. Spheroids are maintained in NC-C medium for 3-4 days as part of a NC maintenance process.

Step 4-EN Induction Phase (Day 15→)

After the ENC spheroid phase (Step 3) and 15 total days from the start of ENC differentiation, ENC spheroids are dissociated with Accutase treatment and replated on PO/LM/FN-coated wells as described in Example 1.

Fluorescence Activated Cell Sorting (FACS)

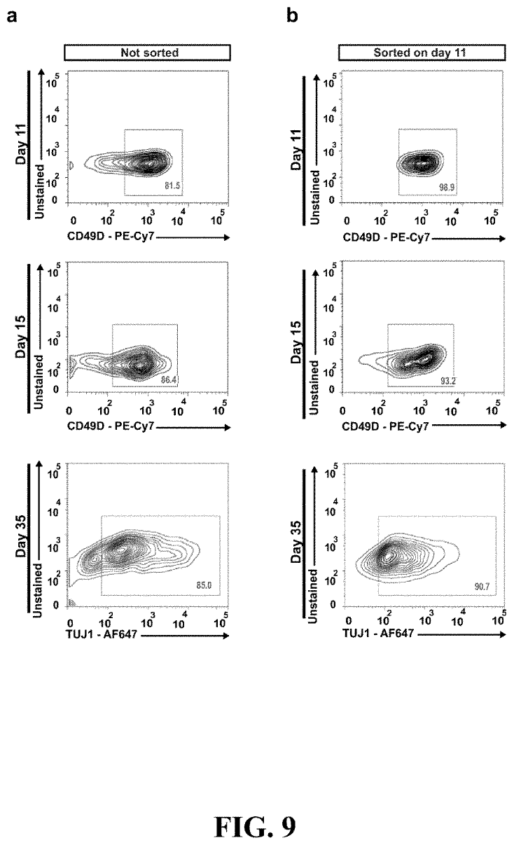

After 12 days of ENC induction under (Step 3), fluorescence activated cell sorting (FACS) can be used to prepare purified populations of NC cells. Previous NC induction protocols have suggested using p75/HNK1 marker staining for FACS analysis^{11,13}. However, p75 expression is found outside of the ENC and a portion of p75/HNK1 double positive cells have been shown to be SOX10::GFP− (12). We have demonstrated that CD49D (α4 integrin) is a specific marker for SOX10+ hPSC-derived NC lineages¹⁶. Here we present a procedure for the purification of ENC cells by FACS using CD49D. FACS purification is particularly recommended for experiments and assays that involve early ENC progenitors (day 11). Further differentiation under the 3D sphere culture condition is generally sufficient to enhance the purity of NC cells and neurons in the later stages of differentiation without FACS purification (FIG. 9).

Reagents
DMEM/F-12, no glutamine (Life Technologies Corporation, 21331020)
BSA, Bovine Serum Albumin (Sigma, A4503)
Anti-human CD49D antibody (Biolegend, 304314)
DAPI (Sigma, D9542)
Normocin, Antimicrobial Reagent (InvivoGen, ant-nr-1)

Equipment
5 ml Round Bottom Polystyrene Test Tube, w/Cell Strainer Cap (Falcon 352235)
5 ml Round Bottom Polystyrene Test Tube, w/Snap Cap (Falcon 352003)
FACS Analyzer (i.e BD LSRFortessa)

Reagent Setup

Staining Medium
Dissolve BSA (0.02 mg ml⁻¹) with DMEM/F-12, no glutamine. Add Pe/Cy7 anti-human CD49D antibody (1.25 μl ml⁻¹). Prepare 2.4 ml per 6-well plate of ENC differentiations (400 μl per well).

Sorting Medium
Dissolve BSA (0.02 mg ml⁻¹) with DMEM/F-12, no glutamine.

Procedure
i. On day 12 of ENC induction, aspirate Cocktail C from ENC induction plate using a sterile pipette tip. Add Accutase (100 μl/cm² well surface area). Incubate at 5% CO₂ and 37° C. for 30 minutes.
ii. DO NOT ASPIRATE Accutase. Use a serological pipet to mechanically harvest cells from the surface of well. Add cell suspension to a 15 ml conical tube.
iii. Centrifuge the conical tube at 1200 rpm (290×g) for 1 minute. With a sterile pipet tip, carefully aspirate as much supernatant as possible while avoiding contact with the cell pellet.
iv. Resuspend the pellet with freshly prepared staining medium (400 μl for every well of a 6-well plate harvested).
v. Place the conical tube of cell suspension in ice for 20 minutes.
vi. After 20 minutes, centrifuge the conical tube at 1200 rpm (290×g) for 1 minute. With a sterile pipet tip, carefully aspirate as much supernatant as possible while avoiding contact with the cell pellet.
vii. Resuspend the pellet with freshly prepared sorting medium (˜1 ml total). Add DAPI (1 μl ml⁻¹).
viii. Transfer the stained cell suspension through the cell strainer cap to a 5 ml round bottom test tube for FACS.
ix. FACS settings may vary per user. Collect CD49D+ population in a sterile 5 ml round bottom test tube and cap. An example of gating strategy is provided in Supplementary FIGS. 6A-6F.
x. Centrifuge the test tube at 1200 rpm (290×g) for 1 minute. With a sterile pipet tip, carefully aspirate as much supernatant as possible while avoiding contact with the cell pellet.
xi. Resuspend the pellet with NC-C (1 ml/10⁶ cells) and transfer suspension to an ultra-low attachment 6-well plate (2 ml/well). Incubate at 37° C. and 5% CO₂.
xii. Resume protocol Step 4-vi.
Note: Sorted cells may be fed with NC-C supplemented with Normocin (1 μl ml⁻¹). Antimicrobial supplemented medium should be used for a minimum of two days.

### Materials

### Reagents—Cell Culture

Human embryonic or induced pluripotent stem cell lines.
The quality of hPSC lines used in your differentiations should be verified by standard characterization of pluripotency including expression of markers such as NANOG and OCT4 and their ability to differentiate into endodermal, mesodermal and ectodermal lineages. The cell lines used in this manuscript are human ES cell line H9 (WA-09) derivative SOX10::GFP (WiCell Research Institute, Memorial Sloan Kettering Cancer Center), human ES cell line UCSF4 (UCSF) and human iPS cell line WTC11 (Coriell Institute, UCSF).
Appropriate consent procedures and administrative regulations must be followed for work involving hESCs and hiPSCs. Please consult your institution to assure adherence with national and institutional guidelines and regulations.
The hPSC lines should be STR profiled to confirm their identity and ensure they are not cross contaminated. Regular karyotyping and frequent mycoplasma testing are necessary to monitor genomic stability and to avoid latent contamination.
DMEM/F-12, no glutamine (Life Technologies, 21331020)
ESSENTIAL 8™ Flex Medium Kit (Life Technologies, A2858501)
ESSENTIAL 6™ Medium (Life Technologies, A1516401)
Neurobasal™ Medium (Life Technologies, 21103049)
N-2 Supplement (CTS™, A1370701)
B-27™ Supplement, serum free (Life Technologies, 17504044)
MEM Nonessential Amino Acids (Corning, 25-025-CI)
GLUTAGRO™ (Corning, 25-015-CI)
BSA, Bovine Serum Albumin (Sigma, A4503)
PBS, Phosphate-Buffered Saline, Ca2+- and Mg2+-free (Life Technologies, 10010023)
EDTA (Corning, MT-46034CI)
ACCUTASE™ (Stemcell Technologies, 07920)
STEM-CELLBANKER® DMSO Free (Amsbio, 11897F)
BMP-4, Recombinant Human BMP-4 Protein (R&D Systems, 314-BP) Stock aliquots should be at stored −80° C. One aliquot should be kept at 4° C. to avoid multiple freeze/thaw cycles and used within 4 weeks.
CHIR 99021 (Tocris, 4423) Stock aliquots should be stored at −20° C. One aliquot should be kept at 4° C. and used within 4 weeks.
FGF2, Recombinant Human FGF Basic (R&D Systems #233-FB) Stock aliquots should be stored at −80° C. One aliquot should be kept at 4° C. to avoid multiple freeze/thaw cycles and used within 4 weeks.
GDNF, Recombinant Human Glial Derived Neurotrophic Factor (Peprotech, 450-10) Stock aliquots should be stored at −80° C. One aliquot should be kept at 4° C. to avoid multiple freeze/thaw cycles and used within 4 weeks.
RA, Retinoic Acid (Sigma, R2625) Stock aliquots should be stored at −80° C. One aliquot should be kept at 4° C. to avoid multiple freeze/thaw cycles and used within 4 weeks.
SB431542 (R&D Systems, 1614) Stock aliquots should be stored at 4° C.
Y-27632 dihydrochloride ((Tocris Bioscience, 1254) Stock aliquots should be stored at −20° C. One aliquot should be kept at 4° C. and used within 4 weeks.
MATRIGEL® hESC-Qualified Matrix, *LDEV-Free, (Corning, 354277)
Vitronectin XF (Stemcell Technologies, 07180)
FN, Fibronectin, Human (Corning, 356008) Stock aliquots should be stored at −80° C. One aliquot should be kept at 4° C. and used within 4 weeks.
LM, Laminin I, Mouse (Cultrex, 3400-010) Stocks should be stored at −80° C.
PO, Poly-L-Ornithine Hydrobromide (Sigma, P3655) Stock aliquots should be stored at −80° C. One aliquot should be kept at 4° C. and used within 4 weeks.
Trypan Blue Solution, 0.4% (Life Technologies, 15250061) Caution: Trypan Blue is a suspected carcinogen and should be handled with care. Collect all materials exposed to Trypan Blue for disposal according to institutional guidelines.
Gelatin, powder (Sigma, G9391)
MEF CF-1 mitomycin C-treated mouse embryonic fibroblasts (Applied StemCell, Inc., ASF-1223)
FBS, fetal bovine serum (Sciencell, 0025)
DMEM, Dulbecco's modified Eagle medium (Life Technologies, 11965-118).
Collagenase IV (Life Technologies, 17104-019)
KSR, Knockout Serum Replacement (Life Technologies, 10828-028)
L-glutamine (Life Technologies, 25030-081)
Knockout DMEM (Life Technologies, 10829-018)
KSR, Knockout Serum Replacement (Life Technologies, 10828-028)
2-mercaptoethanol (Life Technologies, 21985-023)
DMEM/F12 powder (Life Technologies, 12500-062)
Glucose (Sigma, G7021)
Sodium bicarbonate (Sigma, S5761)
Putrescine (Sigma, cat. no. P5780)
Progesterone (Sigma, cat. no. P8783)
Sodium selenite (Bioshop Canada, SEL888)
Transferrin (Celliance/Millipore, 4452-01)
Insulin (Sigma, 16634)

### Reagents—QRT-PCR

RNeasy RNA purification kit (Qiagen, 74106)
SYBR™ Green PCR Master Mix (Applied Biosystems, 4309155)
Superscript IV Reverse Transcriptase Kit (Invitrogen, 18090010)
RNASEOUT™ Recombinant Ribonuclease Inhibitor (Invitrogen, 10777019)
Random Primers (Invitrogen, 48190011)
dNTPs for cDNA Probe Synthesis (10 mM) (Invitrogen, AM8200)
Hs_SOX10_1_SG QuantiTect Primer Assay (Qiagen, QT0005540)
Hs_EDNRB_1_SG QuantiTect Primer Assay (Qiagen, QT00014343)
Hs_PHOX2A_1_SG QuantiTect Primer Assay (Qiagen, QT00215467)
Hs_PHOX2B_1_SG QuantiTect Primer Assay (Qiagen, QT00015078)
Hs_HAND2_2_SG QuantiTect Primer Assay (Qiagen, QT01012907)
Hs_ASCL1_1_SG QuantiTect Primer Assay (Qiagen, QT00237755)
Hs_NTRK3_1_SG QuantiTect Primer Assay (Qiagen, QT00052906)
Hs_ASLC6A4_1_SG QuantiTect Primer Assay (Qiagen, QT00058380)
Hs_CHAT_1_SG QuantiTect Primer Assay (Qiagen, QT00029624)
Hs_SERT_1_SG QuantiTect Primer Assay (Qiagen, QT0058380)
Hs_NOS1_1_SG QuantiTect Primer Assay (Qiagen, QT00043372)
Hs_TUBB_1_SG QuantiTect Primer Assay (Qiagen, QT00089775)
Hs_GFAP_1_SG QuantiTect Primer Assay (Qiagen, QT00081151)
Hs_GAPDH_1_SG QuantiTect Primer Assay (Qiagen, QT00079247)

### Reagents—Immunocytochemistry and Flow Cytometry

PFA, Paraformaldehyde Solution 4% in PBS (Alfa Aesar, J19943K2)
Caution: PFA is a known mutagen and irritant and should be handled with care. Collect all PFA containing solutions for disposal according to institutional guidelines.
Fixation/Permeabilization Solution Kit (BD Biosciences, 554714)
Perm/Wash Buffer (BD PERM/WASH™, 554723)
Pe/Cy7 CD49D antibody (BioLegend, 304314)
Anti-TUJ1 Antibody (Mouse) (BioLegend, 801202)
Anti-Serotonin-5-HT Antibody (Rabbit) (Sigma, S5545)
Anti-GABA Antibody (Rabbit) ((Sigma, S5545)
Anti-NOS1 Antibody (Rabbit) (Santa Cruz Biotechnology, sc648)
Alexa Fluor 488 donkey anti-mouse IgG (Life Technologies, A21202)
Alexa Fluor 647 donkey anti-rabbit IgG (Life Technologies, A31573)
DAPI (Sigma, D9542)
Caution: DAPI is a known mutagen and should be handled with care. Collect all DAPI containing solutions for disposal according to institutional guidelines.

### Equipment

Horizontal Laminar Flow Hood
Cell culture centrifuge (i.e. Eppendorf 5810R)
Inverted microscope (i.e. Evos FL) with fluorescence equipment and digital imaging capture system.
CO₂ incubator with controlling and monitoring system for CO₂, humidity and temperature
Refrigerator 4° C., freezer −20° C., freezer −80° C.
Cell culture disposables: Petri dishes, multiwell plates, conical tubes, pipettes, pipette tips, cell scrapers, etc.
Hemocytometer (i.e. Hausser Scientific)
qPCR System (i.e. 7900HT Fast Real-Time PCR System)
FACS Analyzer (i.e. BD LSRFortessa)

### REFERENCES

1. Shyamala, K., Yanduri, S., Girish, H. C. & Murgod, S. Neural crest: The fourth germ layer. J. Oral Maxillofac. Pathol. 19, 221 (2015).
2. Crane, J. F. & Trainor, P. A. Neural Crest Stem and Progenitor Cells. Annu. Rev. Cell Dev. Biol. 22, 267-286 (2006).
3. Thomas, S. et al. Human neural crest cells display molecular and phenotypic hallmarks of stem cells. Hum. Mol. Genet. 17, 3411-3425 (2008).
4. Chan, K. K. et al. Hoxb3 vagal neural crest-specific enhancer element for controlling enteric nervous system development. Dev. Dyn. 233, 473-483 (2005).
5. Fu, M., Chi Hang Lui, V., Har Sham, M., Nga Yin Cheung, A. & Kwong Hang Tam, P. HOXB5 expression is spatially and temporarily regulated in human embryonic gut during neural crest cell colonization and differentiation of enteric neuroblasts. Dev. Dyn. 228, 1-10 (2003).
6. Heanue, T. A. & Pachnis, V. Enteric nervous system development and Hirschsprung's disease: advances in genetic and stem cell studies. Nat. Rev. Neurosci. 8, 466 (2007).
7. Cheeseman, B. L., Zhang, D., Binder, B. J., Newgreen, D. F. & Landman, K. A. Cell lineage tracing in the developing enteric nervous system: superstars revealed by experiment and simulation. J. R. Soc. Interface 11, 20130815 (2014).
8. Wallace, A. S. & Burns, A. J. Development of the enteric nervous system, smooth muscle and interstitial cells of Cajal in the human gastrointestinal tract. Cell Tissue Res. 319, 367-382 (2005).
9. Kim, J., Lo, L., Dormand, E. & Anderson, D. J. SOX10 maintains multipotency and inhibits neuronal differentiation of neural crest stem cells. Neuron 38, 17-31 (2003).
10. Lasrado, R. et al. Lineage-dependent spatial and functional organization of the mammalian enteric nervous system. Science 356, 722-726 (2017).
11. Lee, G., Chambers, S. M., Tomishima, M. J. & Studer, L. Derivation of neural crest cells from human pluripotent stem cells. Nat. Protoc. 5, 688-701 (2010).
12. Mica, Y., Lee, G., Chambers, S. M., Tomishima, M. J. & Studer, L. Modeling neural crest induction, melanocyte specification, and disease-related pigmentation defects in hESCs and patient-specific iPSCs. Cell Rep. 3, 1140-1152 (2013).
13. Fattahi, F., Studer, L. & Tomishima, M. J. Neural Crest Cells from Dual SMAD Inhibition. Curr. Protoc. Stem Cell Biol. 33, 1H.9.1-9 (2015).
14. Fattahi, F. et al. Deriving human ENS lineages for cell therapy and drug discovery in Hirschsprung disease. Nature 531, 105-109 (2016).
15. Tchieu, J. et al. A Modular Platform for Differentiation of Human PSCs into All Major Ectodermal Lineages. Cell Stem Cell 21, 399-410.e7 (2017).
16. Hackland, J. O. S. et al. Top-Down Inhibition of BMP Signaling Enables Robust Induction of hPSCs Into Neural Crest in Fully Defined, Xeno-free Conditions. Stem Cell Rep. 9, 1043-1052 (2017).
17. Chalazonitis, A. et al. Neurotrophin-3 induces neural crest-derived cells from fetal rat gut to develop in vitro as neurons or glia. J. Neurosci. Off. J. Soc. Neurosci. 14, 6571-6584 (1994).
18. Chalazonitis, A., Rothman, T. P., Chen, J. & Gershon, M. D. Age-dependent differences in the effects of GDNF and NT-3 on the development of neurons and glia from neural crest-derived precursors immunoselected from the fetal rat gut: expression of GFRalpha-1 in vitro and in vivo. Dev. Biol. 204, 385-406 (1998).
19. Memic, F. et al. Transcription and Signaling Regulators in Developing Neuronal Subtypes of Mouse and Human Enteric Nervous System. Gastroenterology (2017). doi:10.1053/j.gastro.2017.10.005
20. Lai, F. P.-L. et al. Correction of Hirschsprung-Associated Mutations in Human Induced Pluripotent Stem Cells Via Clustered Regularly Interspaced Short Palindromic Repeats/Cas9, Restores Neural Crest Cell Function. Gastroenterology 153, 139-153.e8 (2017).
21. Burns, A. J. et al. White paper on guidelines concerning enteric nervous system stem cell therapy for enteric neuropathies. Dev. Biol. 417, 229-251 (2016).
22. Stamp, L. A. Cell therapy for GI motility disorders: comparison of cell sources and proposed steps for treating Hirschsprung disease. Am. J. Physiol.-Gastrointest. Liver Physiol. 312, G348-G354 (2017).
23. Li, W. et al. Characterization and transplantation of enteric neural crest cells from human induced pluripotent stem cells. Mol. Psychiatry 23, 499-508 (2018).
24. Workman, M. J. et al. Engineered human pluripotent-stem-cell-derived intestinal tissues with a functional enteric nervous system. Nat. Med. 23, 49-59 (2017).
25. Gershon, M. D. & Ratcliffe, E. M. Developmental biology of the enteric nervous system: Pathogenesis of Hirschsprung's disease and other congenital dysmotilities. Semin. Pediatr. Surg. 13, 224-235 (2004).
26. Chen, G. et al. Chemically defined conditions for human iPSC derivation and culture. Nat. Methods 8, 424-429 (2011).
27. Claassen, D. A., Desler, M. M. & Rizzino, A. ROCK inhibition enhances the recovery and growth of cryopreserved human embryonic stem cells and human induced pluripotent stem cells. Mol. Reprod. Dev. 76, 722-732 (2009).
28. Maldonado, M., Luu, R. J., Ramos, M. E. P. & Nam, J. ROCK inhibitor primes human induced pluripotent stem cells to selectively differentiate towards mesendodermal lineage via epithelial-mesenchymal transition-like modulation. Stem Cell Res. 17, 222-227 (2016).
29. Sasselli, V., Pachnis, V. & Burns, A. J. The enteric nervous system. Dev. Biol. 366, 64-73 (2012).

1. A method of culturing pluripotent stem cells comprising:
(a) diluting pluripotent stem cells with a culture medium to obtain a pluripotent stem cell mixture;
(b) centrifuging the pluripotent stem cell mixture to obtain a pellet and a supernatant;
(c) removing the supernatant from the pellet;
(d) adding culture medium to the pellet and resuspending the pluripotent stem cells in the culture medium to obtain resuspended pluripotent stem cells;
(e) plating the resuspended pluripotent stem cells on a hydrogel disposed within a culture vessel to obtain plated pluripotent stem cells; and
(f) incubating the plated pluripotent stem cells to a confluency of about 80%.

2. The method of claim 1, wherein, in step (a) or (d), the culture medium is removed and replaced with fresh culture medium about every 2 days.

3. (canceled)

4. The method of claim 1, wherein the pluripotent stem cells are human pluripotent stem cells.

5. The method of claim 1, wherein the pluripotent stem cells are selected from the group consisting of human ES cell line H9 (WA-09), human ES cell line UCSF4, human iPS cell line WTC11, and combinations thereof.

6. The method of claim 1, wherein the hydrogel comprises a solubilized basement membrane preparation extracted from Engelbreth-Holm-Swarm mouse sarcoma, the solubilized basement membrane preparation comprising a laminin, a collagen IV, a heparin sulfate proteoglycan, and entactin/nidogen.

7. The method of claim 1, wherein the hydrogel comprises vitronectin.

8. The method of claim 1, wherein the culture medium comprises a Rho-kinase inhibitor.

9. The method of claim 8, wherein the Rho-kinase inhibitor is Y-27632.

10. The method of claim 8, further comprising in (a) or (d) removing the culture medium comprising the Rho-kinase inhibitor from the culture vessel 3-5 hours after plating and adding E8-C medium without any Rho-kinase inhibitor to the culture vessel.

11. (canceled)

12. The method of claim 1, further comprising passaging the pluripotent stem cells at least twice;
wherein passaging comprises:
washing the pluripotent stem cells to obtain washed pluripotent stem cells;
displacing the washed pluripotent stem cells by adding (ethylenedinitrilo)tetraacetic acid (EDTA) to the culture vessel to obtain displaced pluripotent stem cells;
transferring the displaced pluripotent stem cells to a centrifuge tube;
centrifuging the centrifuge tube comprising the displaced pluripotent stem cells to obtain a second pellet and second supernatant;
separating the second supernatant from the second pellet;
adding culture medium to the centrifuge tube and resuspending the pluripotent stem cells in the second pellet to obtain a second resuspended pluripotent stem cells;
plating the second resuspended pluripotent stem cells to obtain a second plated pluripotent stem cells; and
incubating the second plated pluripotent stem cells to a confluency of about 80%, wherein the culture medium is removed and replaced about every other day.

13. A method of producing an in vitro model of the enteric nervous system comprising:
i. contacting pluripotent stem cells to a first hydrogel disposed in a first culture vessel;
ii. applying a first culture medium into the first culture vessel in a volume sufficient to cover the pluripotent stem cells in contact with the first hydrogel;
iii. incubating the pluripotent stem cells for a first time and under conditions sufficient to grow a confluent layer of pluripotent stem cells;
iv. inducing the pluripotent stem cells for a second time and under conditions sufficient to differentiate the induced pluripotent stem cells into enteric neural crest cells (ENCs);
v. transferring the ENCs to a second culture vessel;
vi. culturing the ENCs for a third time and under conditions for the ENCs to grow into enteric neural crest spheroids; and
vii. contacting the enteric neural crest spheroids to a second hydrogel disposed in a third culture vessel;
viii. applying a second culture medium into the third culture vessel in a volume sufficient to cover the enteric neural crest spheroids in contact with the second hydrogel; and
ix. incubating the enteric neural crest spheroids for a third time and under conditions sufficient to differentiate the enteric neural crest spheroids into enteric neurons;
wherein the ENCs comprise expression of about 5% CD49D and/or sex determining region of the Y chromosome-like high-mobility box transcription factor 10 (SOX10) higher than that expressed by pluripotent stem cells;
wherein the enteric neurons comprise expression of about 5% class III beta-tubulin (TUJ1) and tyrosine-protein receptor kinase C (TRKC) higher than that expressed by ENCs; and
wherein the enteric neurons comprise less than about 60% flat myofibroblast-like cells comprising expression of smooth muscle actin.

14. The method of claim 13, wherein the pluripotent stem cells are human pluripotent stem cells.

15. The method of claim 13, wherein the pluripotent stem cells are comprise one or a combination of: human ES cell line H9 (WA-09), human ES cell line UCSF4, or human iPS cell line WTC11.

16. The method of claim 15, wherein the pluripotent stem cells are human ES cell line UCSF4, and wherein an induction efficiency at day 11 is at least about 25% as measured by expression of CD49D.

17-18. (canceled)

19. The method of claim 16, wherein the induction efficiency at day 15 is at least about 70%.

20.-21. (canceled)

22. The method of claim 15, wherein the pluripotent stem cells are human iPS cell line WTC11, and wherein an induction efficiency at day 11 is at least about 10% as measured by expression of CD49D.

23.-27. (canceled)

28. The method of claim 13, wherein an induction efficiency at day about 20 is at least about 25% as measured by expression of TUJ1 and TRKC.

29-36. (canceled)

37. The method of claim 13, wherein the first and/or second culture medium is E8-C medium.

38. The method of claim 13, wherein iv comprises:
i. removing the first culture medium from the first culture vessel;
ii. adding a first ENC induction medium to the first culture vessel and incubating the differentiating pluripotent stem cells for two days;
iii. removing the first ENC induction medium from the first culture vessel;
iv. adding a second ENC induction medium to the first culture vessel and incubating the differentiating pluripotent stem cells for two days;
v. removing the second ENC induction medium;
vi. replacing the second ENC induction medium with fresh second ENC induction medium and incubating the differentiating pluripotent stem cells for two days;
vii. repeating v and vi;
viii. removing the second ENC induction medium;
ix. adding a third ENC induction medium and incubating the differentiating pluripotent stem cells for two days;
x. removing the third ENC induction medium;
xi. replacing the third ENC induction medium with fresh third ENC induction medium and incubating the differentiating pluripotent stem cells for two days; and
xii. obtaining enteric neural crest cells.

39. The method of claim 38, wherein the first and/or second culture medium is E8-C medium.

40. The method of claim 38, wherein the first induction medium is free of a Smad signaling inhibitor.

41. The method of claim 38, wherein the first induction medium comprises bone morphogenetic protein 4 (BMP4).

42-45. (canceled)

46. The method of claim 38, wherein the ENC comprise expression of at least one of homeobox B2 (HoxB2), homeobox B5 (HoxB5), and paired box 3 (PAX3) at 5% higher than expressed by pluripotent stem cells.

47-48. (canceled)

49. The method of claim 13, wherein the enteric neurons comprise expression of at least one of choline acetyltransferase (CHAT), serotonin (5-HT), gamma-aminobutyric acid (GABA), or neuronal nitric oxide synthase (nNOS).

50-55. (canceled)

56. A system comprising:
a culture vessel comprising a hydrogel;
enteric neurons, wherein the enteric neurons are disposed in a two-dimensional layer on the hydrogel; and
a culture medium, wherein the culture medium is free of any Smad signaling inhibitor,
wherein the enteric neurons are in culture for 5-20 days; and
wherein the enteric neurons comprise less than 60% of cells comprising expression of smooth muscle actin.

57. The system of claim 56, wherein the cells comprising expression of smooth muscle actin are selected from the group consisting of flat myofibroblast like cells and mesenchymal precursors.

58. The system of claim 57, wherein the cells comprising expression of smooth muscle actin are flat myofibroblast like cells.

59. The system of claim 57, wherein the cells comprising expression of smooth muscle actin are mesenchymal precursors.

60. The system of claim 56, wherein the cells comprising expression of smooth muscle actin are a combination of flat myofibroblast like cells and mesenchymal precursors.

61. The system of claim 56, wherein the culture vessel comprises a multi-well plate.

62. The system of claim 56, wherein the hydrogel comprises a solubilized basement membrane preparation extracted from Engelbreth-Holm-Swarm mouse sarcoma, the solubilized basement membrane preparation comprising a laminin, a collagen IV, a heparin sulfate proteoglycan, and entactin/nidogen.

63. The system of claim 56, wherein the hydrogel comprises vitronectin.

64-66. (canceled)

67. A method of differentiating one or a plurality of stem cells into one or a plurality of enteric neuronal cells in a culture vessel comprising a solid substrate, said method comprising:
(a) contacting one or a plurality of stem cells with the solid substrate, said substrate comprising at least one exterior surface, at least one interior surface, and at least one interior chamber defined by the at least one interior surface and accessible from a point exterior to the solid substrate through at least one opening;
(b) applying a first cell medium into the culture vessel for a time period sufficient to differentiate the one or plurality of cells into one or a plurality of neural crest cells;
(c) removing the first cell medium from the culture vessel; and
(d) applying a second cell medium into the culture vessel for a time period sufficient to differentiate the neural crest cells into enteric neurons.

68.-71. (canceled)

72. The method of claim 67, wherein the neural crest cells are exposed to the third cell medium from about 1 to about 3 days before steps (c) and (d).

73. The method of claim 67, wherein the first or second cell medium comprises SB431542, retonic acid or a combination thereof.


SEQUENCE LISTING (18 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 478 aa, Rattus norvegicus
MASLRPFFILALLALVSLADQESCKGRCTQGFMASKKCQCDELCTYYQSCCVDYMEQCKPQVTRGDVFTMPEDEYWSYDYPEETKNSTSTGVQSENTSLHFNLKPRAEETIKPTTPDPQEQSNTQEPEVGQQGVAPRPDTTDEGTSEFPEEELCSGKPFDAFTDLKNGSLFAFRGEYCYELDETAVRPGYPKLIQDVWGIEGPIDAAFTRINCQGKTYLFKGSQYWRFEDGVLDPDYPRNISEGFSGIPDNVDAALALPAHSYSGRERVYFFKGKQYWEYEFQQQPSQEECEGSSLSAVFEHFALLQRDSWENIFELLFWGRSSDGAKGPQFISRDWHGVPGKVDAAMAGRIYITGSTFRSVQAKKQKSGRRSRKRYRSRRGRGHSRSRSRSMSSRRPSRSVWFSLLSSEESGLGTYNYDYDMNWRIPATCEPIQSVYFFSGDKYYRVNLRTRRVDSVNPPYPRSIAQYWLGCPTSEK

SEQ ID NO: 3 - protein, 478 aa, Mus musculus
MAPLRPFFILALVAWVSLADQESCKGRCTQGFMASKKCQCDELCTYYQSCCADYMEQCKPQVTRGDVFTMPEDDYWSYDYVEEPKNNTNTGVQPENTSPPGDLNPRTDGTLKPTAFLDPEEQPSTPAPKVEQQEEILRPDTTDQGTPEFPEEELCSGKPFDAFTDLKNGSLFAFRGQYCYELDETAVRPGYPKLIQDVWGIEGPIDAAFTRINCQGKTYLFKGSQYWRFEDGVLDPGYPRNISEGFSGIPDNVDAAFALPAHRYSGRERVYFFKGKQYWEYEFQQQPSQEECEGSSLSAVFEHFALLQRDSWENIFELLFWGRSSDGAREPQFISRNWHGVPGKVDAAMAGRIYVTGSLSHSAQAKKQKSKRRSRKRYRSRRGRGHRRSQSSNSRRSSRSIWFSLFSSEESGLGTYNNYDYDMDWLVPATCEPIQSVYFFSGDKYYRVNLRTRRVDSVNPPYPRSIAQYWLGCPTSEK

SEQ ID NO: 4 - protein, 748 aa, Homo sapiens
MGLRTAKKRGLGGGGKWKREEGGGTRGRREVRPACFLQSGGRGDPGDVGGPAGNPGCSPHPRAATRPPPLPAHTPAHTPEWCGAASAEAAEPRRAGPHLCIPAPGLTKTPILEKVPRKMAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHVTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 5 - protein, 640 aa, Rattus norvegicus
MPILEKAPQKMPVKASSWEELDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAACLISGVLSYKTLLDSHSLPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGELSDTHRALQLLHGGGCSLNGANRWYDKSLQFVVGRDGTCGVVCEHSPFDGIVLVQCTEHLLKHMMTSNKKLVRADSVSELPAPRRLRLKCSPETQGHLASSAEKLQRIVKNLDFIVYKFDNYGKTFIKKQKYSPDGFIQVALQLAYYRLYQRLVPTYESASIRRFQEGRVDNIRSATPEALAFVQAMTDHKAAMPASEKLQLLQTAMQAHKQYTVMAITGMAIDNHLLALRELARDLCKEPPEMFMDETYLMSNRFVLSTSQVPTTMEMFCCYGPVVPNGNGACYNPQPEAITFCISSFHSCKETSSVEFAEAVGASLVDMRDLCSSRQPADSKPPAPKEKARGPSQAKQS

SEQ ID NO: 6 - protein, 641 aa, Mus musculus
MPILEKVPPKMPVQASSCEEVLDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAASLISGVLSYKALLDSQSIPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGDLSDTHRALQLLHGGGCSLNGANRWYDKSLQFVVGRDGTCGVVCEHSPFDGIVLVQCTEHLLKHMMTGNKKLVRVDSVSELPAPRRLRWKCSPETQGHLASSAEKLQRIVKNLDFIVYKFDNYGKTFIKKQKCSPDGFIQVALQLAYYRLYQRLVPTYESASIRRFQEGRVDNIRSATPEALAFVQAMTDHKAAVLASEKLQLLQRAIQAQTEYTVMAITGMAIDNHLLALRELARDLCKEPPEMFMDETYLMSNRFILSTSQVPTTMEMFCCYGPVVPNGYGACYNPHAEAITFCISSFHGCKETSSVEFAEAVGASLVDMRDLCSSRQPADSKPPTAKERARGPSQAKQS

SEQ ID NO: 7 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 8 - protein, 422 aa, Rattus norvegicus
MDVFSFGQGNNTTASQEPFGTGGNVTSISDVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVRKVEKKGAGTSLGTSSAPPPKKSLNGQPGSGDWRRCAENRAVGTPCTNGAVRQGDDEATLEVIEVHRVGNSKEHLPLPSESGSNSYAPACLERKNERNAEAKRKMALARERKTVKTLGIIMGTFILCWLPFFIVALVLPFCESSCHMPALLGAIINWLGYSNSLLNPVIYAYFNKDFQNAFKKIIKCKFCRR

SEQ ID NO: 9 - protein, 421 aa, Mus musculus
MDMFSLGQGNNTTTSLEPFGTGGNDTGLSNVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSNPNECTISKDHGYTIYSTFGAFYIPLLLMLVLYGRIFRAARFRIRKTVKKVEKKGAGTSFGTSSAPPPKKSLNGQPGSGDCRRSAENRAVGTPCANGAVRQGEDDATLEVIEVHRVGNSKGHLPLPSESGATSYVPACLERKNERTAEAKRKMALARERKTVKTLGIIMGTFILCWLPFFIVALVLPFCESSCHMPELLGAIINWLGYSNSLLNPVIYAYFNKDFQNAFKKIIKCKFCR

SEQ ID NO: 10 - protein, 1434 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRFLKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 11 - protein, 1429 aa, Homo sapiens
MEENTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGPPTKAVDLSHQPSASKDQSLAVDRVTGLGNGPQHAQGHGQGAGSVSQANGVAIDPTMKSTKANLQDIGEHDELLKEIEPVLSILNSGSKATNRGGPAKAEMKDTGIQVDRDLDGKSHKAPPLGGDNDRVFNDLWGKDNVPVILNNPYSEKEQSPTSGKQSPTKNGSPSRCPRFLKVKNWETDVVLTDTLHLKSTLETGCTEHICMGSIMLPSQHTRKPEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKAPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKPNNSLISNDRSWKRNKFRLTYVAEAPDLTQGLSNVHKKRVSAARLLSRQNLQSPKFSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDADEVFSS

SEQ ID NO: 12 - protein, 1429 aa, Homo sapiens
MEEHTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGTPTKAVDLSRQPSASKDQPLAVDRVPGPSNGPQHAQGRGQGAGSVSQANGVAIDPTMKNTKANLQDSGEQDELLKEIEPVLSILTGGGKAVNRGGPAKAEMKDTGIQVDRDLDGKLHKAPPLGGENDRVFNDLWGKGNVPVVLNNPYSENEQSPASGKQSPTKNGSPSRCPRFLKVKNWETDVVLTDTLHLKSTLETGCTEQICMGSIMLPSHHIRKSEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVEFTEICIQQGWKPPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTYVAEAPELTQGLSNVHKKRVSAARLLSRQNLQSPKSSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDTDEVFSS

SEQ ID NO: 13 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 14 - protein, 430 aa, Rattus norvegicus
MERRRITSARRSYASSETMVRGHGPTRHLGTIPRLSLSRMTPPLPARVDFSLAGALNAGFKETRASERAEMMELNDRFASYIEKVRFLEQQNKALAAELNQLRAKEPTKLADVYQAELRELRLRLDQLTTNSARLEVERDNLTQDLGTLRQKLQDETNLRLEAENNLAVYRQEADEATLARVDLERKVESLEEEIQFLRKIHEEEVRELQEQLAQQQVHVEMDVAKPDLTAALREIRTQYEAVATSNMQETEEWYRSKFADLTDVASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 15 - protein, 430 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 16 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 17 - protein, 466 aa, Rattus norvegicus
MAEEQDLSEVELSPVGSEEPRCLSPSSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGETDQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLSSALAVASGHSAWISKPPGVALPTVSPPAVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 18 - protein, 466 aa, Mus musculus
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLGSALAVASGHSAWISKPPGVALPTVSPPGVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP